(19) United States
(12) Patent Application Publication
(10) Pub. No.: US 2010/0016366 A1
(43) Pub. Date: Jan. 21, 2010

(54) Novel Compounds Active as Muscarinic Receptor Antagonists

(76) Inventors: Paul Alan Glossop, Sandwich (GB); Charlotte Alice Louise Lane, Sandwich (GB)

Correspondence Address: PFIZER INC.;PATENT DEPARTMENT, Bld 114 M/S 114, EASTERN POINT ROAD, GROTON, CT, 06340, US

(21) Appl. No.: 12/496,001

(22) Filed: Jul. 1, 2009

Related U.S. Application Data
(60) Provisional application No. 61/080,726, filed on Jul. 15, 2008.

(51) Int. Cl. A61K 31/445; C07D 211/94

(52) U.S. Cl. 514/327; 546/222

(57) ABSTRACT

The invention relates to compounds of formula

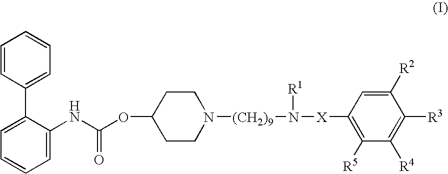

processes and intermediates for their preparation, their use as muscarinic antagonists and pharmaceutical compositions containing them.

This invention relates to compounds of general formula (I):

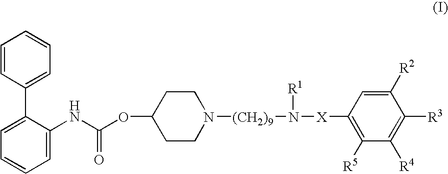

in which R¹ to R⁵ and X have the meanings indicated below, and to processes and intermediates for the preparation of, compositions containing and the uses of such derivatives.

Cholinergic muscarinic receptors are members of the G-protein coupled receptor super-family and are further divided into 5 subtypes, M₁ to M₅. Muscarinic receptor sub-types are widely and differentially expressed in the body. Genes have been cloned for all 5 sub-types and of these, M₁, M₂ and M₃ receptors have been extensively pharmacologically characterized in animal and human tissue. M₁ receptors are expressed in the brain (cortex and hippocampus), glands and in the ganglia of sympathetic and parasympathetic nerves. M₂ receptors are expressed in the heart, hindbrain, smooth muscle and in the synapses of the autonomic nervous system. M₃ receptors are expressed in the brain, glands and smooth muscle. In the airways, stimulation of M₃ receptors evokes contraction of airway smooth muscle leading to bronchoconstriction, while in the salivary gland M₃ receptor stimulation increases fluid and mucus secretion leading to increased salivation. M₂ receptors expressed on smooth muscle are understood to be pro-contractile while pre-synaptic M₂ receptors modulate acetylcholine release from parasympathetic nerves. Stimulation of M₂ receptors expressed in the heart produces bradycardia.

Short and long-acting muscarinic antagonists are used in the management of asthma and COPD; these include the short acting agents Atrovent® (ipratropium bromide) and Oxivent® (oxitropium bromide) and the long acting agent Spiriva® (tiotropium bromide). These compounds produce bronchodilation following inhaled administration. In addition to improvements in spirometric values, anti-muscarinic use in chronic obstructive pulmonary disease (COPD) is associated with improvements in health status and quality of life scores.

As a consequence of the wide distribution of muscarinic receptors in the body, significant systemic exposure to muscarinic antagonists is associated with effects such as dry mouth, constipation, mydriasis, urinary retention (all predominantly mediated via blockade of M₃ receptors) and tachycardia (mediated by blockade of M₂ receptors). A commonly reported side-effect following inhaled administration of therapeutic dose of the current, clinically used non-selective muscarinic antagonists is dry-mouth and while this is reported as only mild in intensity it does limit the dose of inhaled agent given.

Accordingly, there is still a need for M₃ receptor antagonists that would have an appropriate pharmacological profile, for example in term of potency, pharmacokinetics or duration of action and in particular for an administration by the inhalation route. In addition, as muscarinic receptor antagonists are suitable for the treatment of chronic diseases, such as asthma or COPD, they are likely to be co-administered with other compounds at least from time to time. Thus, such compounds would preferably have a low potential for interaction with co-administered compounds. In this context, the present invention relates to novel M₃ receptor antagonists.

The invention relates to a compound of formula (I)

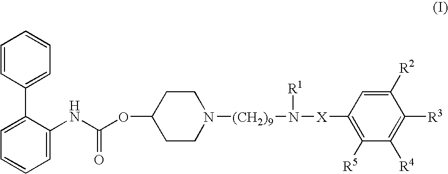

or the pharmaceutically acceptable salts thereof, or the pharmaceutically acceptable solvates of said compounds or salts, wherein:
X is selected from —CH₂—, —C(═O)CH₂—, —C(═O)—;
R¹ is H or methyl or alternatively when X is —CH₂— then R¹ can also represent a group of formula:

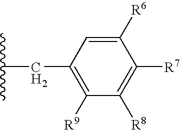

wherein one of R⁶, R⁷, R⁸ and R⁹ is OH, one of R⁶, R⁷, R⁸ and R⁹ is halo, one of R⁶, R⁷, R⁸ and R⁹ is H, and one of R⁶, R⁷, R⁸ and R⁹ is selected from H or halo;
one of R², R³, R⁴ and R⁵ is OH, one of R², R³, R⁴ and R⁵ is H, one of R², R³, R⁴ and R⁵ is halo, and one of R², R³, R⁴ and R⁵ is H or halo, or alternatively when X is —C(═O)CH₂— and R¹ is methyl then R⁴ can also be OH while R², R³ and R⁵ are H.

In the here above general formula (I), the term “halo” denotes a halogen atom selected from the group consisting of fluoro, chloro, bromo and iodo. Preferred halo groups are fluoro or chloro.

Preferred compounds according to the invention are:
Biphenyl-2-yl-carbamic acid 1-[9-(3-chloro-4-hydroxy-benzoylamino)-nonyl]-piperidin-4-yl ester;
Biphenyl-2-yl-carbamic acid 1-[9-(2-fluoro-3-hydroxy-benzylamino)-nonyl]-piperidin-4-yl ester;
Biphenyl-2-yl-carbamic acid 1-[9-(3-chloro-5-fluoro-2-hydroxy-benzylamino)-nonyl]-piperidin-4-yl ester;
Biphenyl-2-yl-carbamic acid 1-{9-[2-(3-chloro-4-hydroxy-phenyl)-acetylamino]-nonyl}-piperidin-4-yl ester;
Biphenyl-2-yl-carbamic acid 1-[9-(2-chloro-3-hydroxy-benzylamino)-nonyl]-piperidin-4-yl ester;
Biphenyl-2-yl-carbamic acid 1-[9-(3-fluoro-4-hydroxy-benzoylamino)-nonyl]-piperidin-4-yl ester;
Biphenyl-2-yl-carbamic acid 1-[9-(4-fluoro-3-hydroxy-benzoylamino)-nonyl]-piperidin-4-yl ester;
Biphenyl-2-yl-carbamic acid 1-[9-(3-fluoro-2-hydroxy-benzoylamino)-nonyl]-piperidin-4-yl ester;
Biphenyl-2-yl-carbamic acid 1-[9-(5-fluoro-2-hydroxy-benzoylamino)-nonyl]-piperidin-4-yl ester;
Biphenyl-2-yl-carbamic acid 1-[9-(3,5-dichloro-2-hydroxy-benzoylamino)-nonyl]-piperidin-4-yl ester;
Biphenyl-2-yl-carbamic acid 1-[9-(4-chloro-2-hydroxy-benzoylamino)-nonyl]-piperidin-4-yl ester;
Biphenyl-2-yl-carbamic acid 1-(9-{[2-(3-chloro-4-hydroxy-phenyl)-acetyl]-methyl-amino}-nonyl)-piperidin-4-yl ester;
Biphenyl-2-yl-carbamic acid 1-{9-[(4-fluoro-3-hydroxy-benzoyl)-methyl-amino]-nonyl}-piperidin-4-yl ester;
Biphenyl-2-yl-carbamic acid 1-{9-[(3-chloro-4-hydroxy-benzoyl)-methyl-amino]-nonyl}-piperidin-4-yl ester;
Biphenyl-2-yl-carbamic acid 1-(9-{[2-(3-fluoro-4-hydroxy-phenyl)-acetyl]-methyl-amino}-nonyl)-piperidin-4-yl ester;
Biphenyl-2-yl-carbamic acid 1-[9-(4-chloro-3-hydroxy-benzoylamino)-nonyl]-piperidin-4-yl ester;
Biphenyl-2-yl-carbamic acid 1-[9-(2-fluoro-4-hydroxy-benzoylamino)-nonyl]-piperidin-4-yl ester;
Biphenyl-2-yl-carbamic acid 1-[9-(3,5-dichloro-4-hydroxy-benzylamino)-nonyl]-piperidin-4-yl ester;
Biphenyl-2-yl-carbamic acid 1-[9-(3,5-dichloro-4-hydroxy-benzylamino)-nonyl]-piperidin-4-yl ester;
naphthalene-1,5-disulfonate salt;
Biphenyl-2-yl-carbamic acid 1-[9-(2,3-difluoro-4-hydroxy-benzoylamino)-nonyl]-piperidin-4-yl ester;
Biphenyl-2-yl-carbamic acid 1-[9-(2-hydroxy-5-chloro-benzoylamino)-nonyl]-piperidin-4-yl ester;
Biphenyl-2-yl-carbamic acid 1-[9-(4-hydroxy-3,5-dichloro-benzoylamino)-nonyl]-piperidin-4-yl ester;
Biphenyl-2-yl-carbamic acid 1-[9-(2-hydroxy-4-fluoro-benzoylamino)-nonyl]-piperidin-4-yl ester;
Biphenyl-2-yl-carbamic acid 1-[9-(3,5-difluoro-4-hydroxy-benzylamino)-nonyl]-piperidin-4-yl ester;
Biphenyl-2-yl-carbamic acid 1-[9-(2-hydroxy-4,5-dichloro-benzylamino)-nonyl]-piperidin-4-yl ester;
Biphenyl-2-yl-carbamic acid 1-[9-(2-hydroxy-3,5-difluoro-benzylamino)-nonyl]-piperidin-4-yl ester;
Biphenyl-2-yl-carbamic acid 1-[9-(2-hydroxy-3-fluoro-benzylamino)-nonyl]-piperidin-4-yl ester;
Biphenyl-2-yl-carbamic acid 1-[9-(2-hydroxy-3,5-dichloro-benzylamino)-nonyl]-piperidin-4-yl ester;
Biphenyl-2-yl-carbamic acid 1-[9-(5-chloro-2-hydroxy-benzylamino)-nonyl]-piperidin-4-yl ester;
Biphenyl-2-yl-carbamic acid 1-[9-(2-hydroxy-4-chloro-5-fluoro-benzylamino)-nonyl]-piperidin-4-yl ester;
Biphenyl-2-yl-carbamic acid 1-[9-(3-chloro-4-hydroxy-benzylamino)-nonyl]-piperidin-4-yl ester;
Biphenyl-2-yl-carbamic acid 1-[9-(2-hydroxy-5-fluoro-benzylamino)-nonyl]-piperidin-4-yl ester;
Biphenyl-2-yl-carbamic acid 1-[9-(3-hydroxy-4-fluoro-benzylamino)-nonyl]-piperidin-4-yl ester;
Biphenyl-2-yl-carbamic acid 1-[9-(2,4-dichloro-3-hydroxy-benzylamino)-nonyl]-piperidin-4-yl ester;
Biphenyl-2-yl-carbamic acid 1-[9-(4-hydroxy-3-fluoro-benzylamino)-nonyl]-piperidin-4-yl ester;
Biphenyl-2-yl-carbamic acid 1-{9-[bis-(2-chloro-3-hydroxy-benzyl)-amino]-nonyl}-piperidin-4-yl ester;
Biphenyl-2-yl-carbamic acid 1-{9-[(3-fluoro-2-hydroxy-benzyl)-methyl-amino]-nonyl}-piperidin-4-yl ester;
Biphenyl-2-yl-carbamic acid 1-{9-[(4,5-dichloro-2-hydroxy-benzyl)-methyl-amino]-nonyl}-piperidin-4-yl ester;
Biphenyl-2-yl-carbamic acid 1-{9-[(4-fluoro-3-hydroxy-benzyl)-methyl-amino]-nonyl}-piperidin-4-yl ester;
Biphenyl-2-yl-carbamic acid 1-{9-[(4-chloro-5-fluoro-2-hydroxy-benzyl)-methyl-amino]-nonyl}-piperidin-4-yl ester;
Biphenyl-2-yl-carbamic acid 1-{9-[(3-chloro-4-hydroxy-benzyl)-methyl-amino]-nonyl}-piperidin-4-yl ester;
Biphenyl-2-yl-carbamic acid 1-{9-[(3-fluoro-4-hydroxy-benzyl)-methyl-amino]-nonyl}-piperidin-4-yl ester;
Biphenyl-2-yl-carbamic acid 1-{9-[(5-chloro-2-hydroxy-benzyl)-methyl-amino]-nonyl}-piperidin-4-yl ester;
Biphenyl-2-yl-carbamic acid 1-{9-[(2-chloro-3-hydroxy-benzyl)-methyl-amino]-nonyl}-piperidin-4-yl ester;
Biphenyl-2-yl-carbamic acid 1-{9-[(3,5-dichloro-4-hydroxy-benzyl)-methyl-amino]-nonyl}-piperidin-4-yl ester;
Biphenyl-2-yl-carbamic acid 1-{9-[(2-fluoro-3-hydroxy-benzyl)-methyl-amino]-nonyl}-piperidin-4-yl ester;
Biphenyl-2-yl-carbamic acid 1-{9-[(3,5-difluoro-4-hydroxy-benzyl)-methyl-amino]-nonyl}-piperidin-4-yl ester;
Biphenyl-2-yl-carbamic acid 1-{9-[(2,4-dichloro-3-hydroxy-benzyl)-methyl-amino]-nonyl}-piperidin-4-yl ester;
Biphenyl-2-yl-carbamic acid 1-{9-[(3,5-difluoro-2-hydroxy-benzyl)-methyl-amino]-nonyl}-piperidin-4-yl ester;
Biphenyl-2-yl-carbamic acid 1-{9-[(3,5-dichloro-2-hydroxy-benzoyl)-methyl-amino]-nonyl}-piperidin-4-yl ester;
Biphenyl-2-yl-carbamic acid 1-{9-[(3-chloro-2-hydroxy-benzoyl)-methyl-amino]-nonyl}-piperidin-4-yl ester;
Biphenyl-2-yl-carbamic acid 1-{9-[(3,4-difluoro-2-hydroxy-benzoyl)-methyl-amino]-nonyl}-piperidin-4-yl ester;
Biphenyl-2-yl-carbamic acid 1-{9-[(3-fluoro-2-hydroxy-benzoyl)-methyl-amino]-nonyl}-piperidin-4-yl ester;
Biphenyl-2-yl-carbamic acid 1-{9-[(4-chloro-2-hydroxy-benzoyl)-methyl-amino]-nonyl}-piperidin-4-yl ester;
Biphenyl-2-yl-carbamic acid 1-{9-[(4-fluoro-2-hydroxy-benzoyl)-methyl-amino]-nonyl}-piperidin-4-yl ester;
Biphenyl-2-yl-carbamic acid 1-{9-[(4-chloro-3-hydroxy-benzoyl)-methyl-amino]-nonyl}-piperidin-4-yl ester;
Biphenyl-2-yl-carbamic acid 1-{9-[(2,3-difluoro-4-hydroxy-benzoyl)-methyl-amino]-nonyl}-piperidin-4-yl ester;
Biphenyl-2-yl-carbamic acid 1-(9-{[2-(3-hydroxy-phenyl)-acetyl]-methyl-amino}-nonyl)-piperidin-4-yl ester;
Biphenyl-2-yl-carbamic acid 1-{9-[(5-fluoro-2-hydroxy-benzoyl)-methyl-amino]-nonyl}-piperidin-4-yl ester;
Biphenyl-2-yl-carbamic acid 1-{9-[(5-chloro-2-hydroxy-benzoyl)-methyl-amino]-nonyl}-piperidin-4-yl ester
   and
Biphenyl-2-yl-carbamic acid 1-{9-[(3-fluoro-4-hydroxy-benzoyl)-methyl-amino]-nonyl}-piperidin-4-yl ester;
   or the pharmaceutically acceptable salts thereof, or the pharmaceutically acceptable solvates of said compounds or salts.

More preferred compounds according to the present invention are:
Biphenyl-2-yl-carbamic acid 1-[9-(3-chloro-4-hydroxy-benzoylamino)-nonyl]-piperidin-4-yl ester;
Biphenyl-2-yl-carbamic acid 1-[9-(2-fluoro-3-hydroxy-benzylamino)-nonyl]-piperidin-4-yl ester;
Biphenyl-2-yl-carbamic acid 1-[9-(3-chloro-5-fluoro-2-hydroxy-benzylamino)-nonyl]-piperidin-4-yl ester;
Biphenyl-2-yl-carbamic acid 1-{9-[2-(3-chloro-4-hydroxy-phenyl)-acetylamino]-nonyl}-piperidin-4-yl ester;
Biphenyl-2-yl-carbamic acid 1-[9-(2-chloro-3-hydroxy-benzylamino)-nonyl]-piperidin-4-yl ester;
Biphenyl-2-yl-carbamic acid 1-[9-(3-fluoro-4-hydroxy-benzoylamino)-nonyl]-piperidin-4-yl ester;
Biphenyl-2-yl-carbamic acid 1-[9-(4-fluoro-3-hydroxy-benzoylamino)-nonyl]-piperidin-4-yl ester;
Biphenyl-2-yl-carbamic acid 1-(9-{[2-(3-chloro-4-hydroxy-phenyl)-acetyl]-methyl-amino}-nonyl)-piperidin-4-yl ester;
Biphenyl-2-yl-carbamic acid 1-{9-[(4-fluoro-3-hydroxy-benzoyl)-methyl-amino]-nonyl}-piperidin-4-yl ester;
Biphenyl-2-yl-carbamic acid 1-{9-[(3-chloro-4-hydroxy-benzoyl)-methyl-amino]-nonyl}-piperidin-4-yl ester;
Biphenyl-2-yl-carbamic acid 1-(9-{[2-(3-fluoro-4-hydroxy-phenyl)-acetyl]-methyl-amino}-nonyl)-piperidin-4-yl ester;
Biphenyl-2-yl-carbamic acid 1-[9-(4-chloro-3-hydroxy-benzoylamino)-nonyl]-piperidin-4-yl ester;
Biphenyl-2-yl-carbamic acid 1-[9-(2-fluoro-4-hydroxy-benzoylamino)-nonyl]-piperidin-4-yl ester;
Biphenyl-2-yl-carbamic acid 1-[9-(3,5-dichloro-4-hydroxy-benzylamino)-nonyl]-piperidin-4-yl ester;
Biphenyl-2-yl-carbamic acid 1-[9-(2,3-difluoro-4-hydroxy-benzoylamino)-nonyl]-piperidin-4-yl ester;
Biphenyl-2-yl-carbamic acid 1-[9-(3,5-difluoro-4-hydroxy-benzylamino)-nonyl]-piperidin-4-yl ester;
Biphenyl-2-yl-carbamic acid 1-[9-(2-hydroxy-3,5-difluoro-benzylamino)-nonyl]-piperidin-4-yl ester;
Biphenyl-2-yl-carbamic acid 1-[9-(2-hydroxy-3,5-dichloro-benzylamino)-nonyl]-piperidin-4-yl ester;
Biphenyl-2-yl-carbamic acid 1-[9-(3-chloro-4-hydroxy-benzylamino)-nonyl]-piperidin-4-yl ester;
Biphenyl-2-yl-carbamic acid 1-[9-(3-hydroxy-4-fluoro-benzylamino)-nonyl]-piperidin-4-yl ester;
Biphenyl-2-yl-carbamic acid 1-[9-(2,4-dichloro-3-hydroxy-benzylamino)-nonyl]-piperidin-4-yl ester;
Biphenyl-2-yl-carbamic acid 1-{9-[bis-(2-chloro-3-hydroxy-benzyl)-amino]-nonyl}-piperidin-4-yl ester;
Biphenyl-2-yl-carbamic acid 1-{9-[(4,5-dichloro-2-hydroxy-benzyl)-methyl-amino]-nonyl}-piperidin-4-yl ester;
Biphenyl-2-yl-carbamic acid 1-{9-[(4-fluoro-3-hydroxy-benzyl)-methyl-amino]-nonyl}-piperidin-4-yl ester;
Biphenyl-2-yl-carbamic acid 1-{9-[(2-chloro-3-hydroxy-benzyl)-methyl-amino]-nonyl}-piperidin-4-yl ester;
Biphenyl-2-yl-carbamic acid 1-{9-[(3,5-dichloro-4-hydroxy-benzyl)-methyl-amino]-nonyl}-piperidin-4-yl ester;
Biphenyl-2-yl-carbamic acid 1-{9-[(3,5-difluoro-4-hydroxy-benzyl)-methyl-amino]-nonyl}-piperidin-4-yl ester;
Biphenyl-2-yl-carbamic acid 1-{9-[(4-chloro-3-hydroxy-benzoyl)-methyl-amino]-nonyl}-piperidin-4-yl ester;
Biphenyl-2-yl-carbamic acid 1-{9-[(2,3-difluoro-4-hydroxy-benzoyl)-methyl-amino]-nonyl}-piperidin-4-yl ester;
Biphenyl-2-yl-carbamic acid 1-(9-{[2-(3-hydroxy-phenyl)-acetyl]-methyl-amino}-nonyl)-piperidin-4-yl ester;
   and
Biphenyl-2-yl-carbamic acid 1-{9-[(3-fluoro-4-hydroxy-benzoyl)-methyl-amino]-nonyl}-piperidin-4-yl ester;
   or the pharmaceutically acceptable salts thereof, or the pharmaceutically acceptable solvates of said compounds or salts.

Even more preferred compounds according to the present inventions are:
Biphenyl-2-yl-carbamic acid 1-(9-{[2-(3-chloro-4-hydroxy-phenyl)-acetyl]-methyl-amino}-nonyl)-piperidin-4-yl ester;
Biphenyl-2-yl-carbamic acid 1-{9-[(3-chloro-4-hydroxy-benzoyl)-methyl-amino]-nonyl}-piperidin-4-yl ester;
Biphenyl-2-yl-carbamic acid 1-[9-(2,4-dichloro-3-hydroxy-benzylamino)-nonyl]-piperidin-4-yl ester;
Biphenyl-2-yl-carbamic acid 1-{9-[(2-chloro-3-hydroxy-benzyl)-methyl-amino]-nonyl}-piperidin-4-yl ester;
Biphenyl-2-yl-carbamic acid 1-{9-[(2,3-difluoro-4-hydroxy-benzoyl)-methyl-amino]-nonyl}-piperidin-4-yl ester;
Biphenyl-2-yl-carbamic acid 1-(9-{[2-(3-hydroxy-phenyl)-acetyl]-methyl-amino}-nonyl)-piperidin-4-yl ester
   and
Biphenyl-2-yl-carbamic acid 1-{9-[(3-fluoro-4-hydroxy-benzoyl)-methyl-amino]-nonyl}-piperidin-4-yl ester;
   or the pharmaceutically acceptable salts thereof, or the pharmaceutically acceptable solvates of said compounds or salts.

Most preferred compounds are Biphenyl-2-yl-carbamic acid 1-(9-{[2-(3-hydroxy-phenyl)-acetyl]-methyl-amino}-nonyl)-piperidin-4-yl ester; and Biphenyl-2-yl-carbamic acid 1-{9-[(3-fluoro-4-hydroxy-benzoyl)-methyl-amino]-nonyl}-piperidin-4-yl ester, or the pharmaceutically acceptable salts thereof, or the pharmaceutically acceptable solvates of said compounds or salts.

Compounds of formula (I) may be prepared in a variety of ways. The routes below illustrate one such way of preparing these compounds; the skilled person will appreciate that other routes may be equally as practicable.

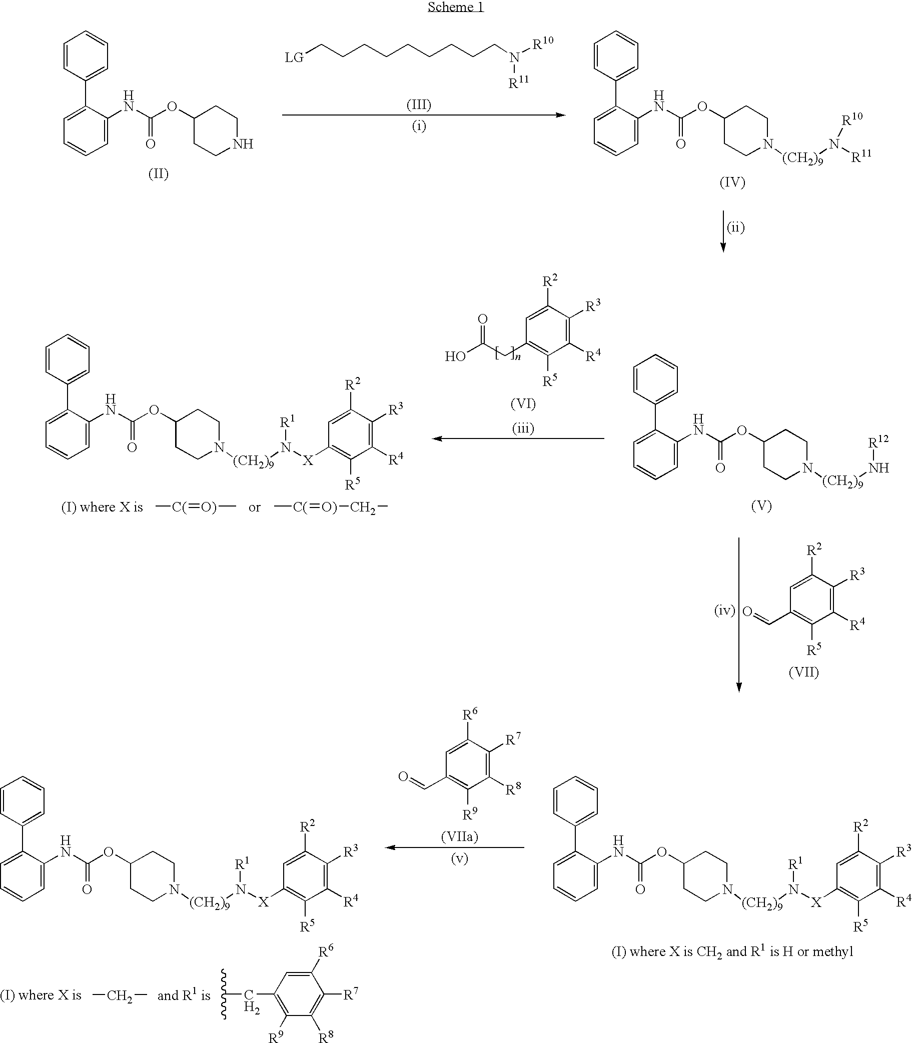

wherein:
R¹⁰ is methyl or a suitable protecting group such as tert-butoxycarbonyl;
R¹¹ is a suitable protecting group such as tert-butoxycarbonyl;
R¹⁰ and R¹¹ may form together a suitable protecting group such as phthalimide;
R¹² is H or methyl;
n=0 or 1;
LG represents a suitable leaving group such as bromide or mesylate; and
R¹ to R⁹ and X are as defined for compounds of formula (I) unless otherwise stated.

The compound of formula (II) may be prepared as described in US 2006/205779.

The compound of formula (III) where LG is bromide and both R¹⁰ and R¹¹ are tert-butoxycarbonyl may be prepared as described in WO 2007/107828.

The compound of formula (III) where LG is mesylate, R¹⁰ is methyl and R¹¹ is tert-butoxycarbonyl may be prepared as described in Scheme 2:

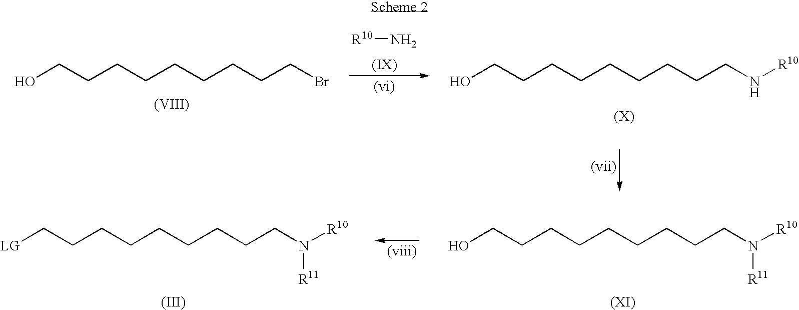

wherein R¹⁰ is methyl, R¹¹ is tert-butoxycarbonyl and LG is mesylate.

The compound of formula (VIII) is commercially available.

The compound of formula (IX) is commercially available.

The compound of formula (X) may be prepared from the compound of formula (VIII) and (IX) by bromide displacement (process step (vi)). Typical conditions comprise reaction of compound (VIII) with excess compound (IX) (methylamine, 33% solution in ethanol) at room temperature for 18 hours.

The compound of formula (XI) may be prepared from the compound of formula (X) by Boc protection (process step (vii)). Typical conditions comprise reaction of compound (X) with Boc anhydride in a suitable solvent such as dichloromethane at 0° C. to room temperature for 4 hours.

The compound of formula (III) may be prepared from the compound of formula (XI) by mesylation (process step (viii)). Typical conditions comprise reaction of compound (XI) with methane sulfonyl chloride and a suitable base such as triethylamine, in a suitable solvent such as dichloromethane, at 5° C. to room temperature for 1 hour.

The compound of formula (III) wherein LG is bromide and R¹⁰ is methyl or wherein LG is mesylate and R¹⁰ is tert-butoxycarbonyl or wherein LG is mesylate or bromide and R¹⁰ and R¹¹ are together a phthalimide may be prepared using a procedure similar to those described in Scheme 2.

The compound of formula (IV) may be prepared from the compound of formula (II) and compound of formula (III), by alkylation (process step (i)). Typical conditions comprise reaction of compound (II) with compound (III) and a suitable base such as triethylamine, sodium carbonate or potassium carbonate, in a suitable solvent such as dimethylformamide, at temperatures between 60-70° C., for 18-48 hours.

The Compounds of formula (V) may be prepared from the compounds of formula (IV), by deprotection using standard methodology as described in “Protecting Groups in Organic Synthesis” by T. W. Greene and P. Wutz (process step (ii)). When R¹⁰ and R¹¹, or R¹¹ is tert-butoxycarbonyl; typical conditions comprise reaction of compound (IV) with hydrogen chloride in a suitable solvent such as dioxane, at room temperature, for 18 hours. When R¹⁰ and R¹¹ represent phthalimide; typical conditions comprise reaction of compound (IV) with hydrazine hydrate in a suitable solvent such as ethanol, at 90° C. for 3 hours.

The compounds of formula (VI) are commercially available, known in the literature or they may be easily prepared by persons skilled in the art according to standard procedures.

The Compounds of formula (VII) and (VIIa) are commercially available, known in the literature or they may be easily prepared by persons skilled in the art according to standard procedures.

The compounds of formula (I) where X is —C(═O)CH₂— or —C(═O)— may be prepared from the compounds of formula (V) and compounds of formula (VI) by acylation (process step (iii)). Typical conditions comprise reaction of compound (V) and compound (VI) with suitable coupling agents such as (3-(dimethylamino)propyl)ethyl carbodiimide hydrochloride or O-(1H-benzotriazol-1-yl)-N,N,N′,N′-tetramethyluronium hexafluorophosphate, optionally in the presence of a suitable additive such as 1-hydroxy benzotriazole monohydrate or N,N-dimethylaminopyridine, with a suitable base such as triethylamine or N,N-diisopropylethylamine, in a suitable solvent such as dichloromethane, dimethylformamide, tetrahydrofuran, 1-methyl-2-pyrrolidinone or 2-methyltetrahydrofuran, at room temperature to 60° C., for 18 to 72 hours.

The compounds of formula (I) where X is CH₂ and R¹ is H or methyl may be prepared from the compounds of formula (V) and compounds of formula (VII) by reductive amination (process step (iv)). Typical conditions comprise reaction of compound (V) with compound (VII) in a suitable solvent such as ethanol, dichloromethane or dichloroethane, optionally in the presence of a suitable catalyst such as acetic acid or titanium tetraisopropoxide, optionally in the presence of a drying agent such as sodium sulfate, and optionally in the presence of a suitable base such as triethylamine, at room temperature for 1 to 18 hours, followed by addition of a suitable reducing agent such as sodium borohydride, sodium triacetoxyborohydride or sodium cyanoborohydride at 0° C. to room temperature, for 18 to 24 hours.

The compounds of formula (I) where X is CH₂ and R¹ is

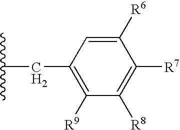

may be prepared from compound of formula (I) where X is CH₂ and R¹ is H and compound of formula (VIIa) by reductive amination (process step (v)). Typical conditions comprise reaction of compound (I) with compound (VIIa) in a similar manner to that previously described for process step (iv).

The preparation of compounds of formula (I) may require the protection of potential reactive functionality in addition to those methods already described. In such a case, examples of compatible protecting groups and their particular methods of protection and deprotection are described in “Protecting Groups in Organic Synthesis” by T. W. Greene and P. Wutz (Wiley-Interscience Publication, 1981) or “Protecting groups” by P. J. Kocienski (Georg Thieme Verlag, 1994).

The compounds of formula (I) as well as intermediates for their preparation can be purified and isolated according to various well-known methods, for example crystallisation or chromatography.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 1,5-naphthalenedisulfonate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘Iyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) include references to salts, solvates, multi-component complexes and liquid crystals thereof and to solvates, multi-component complexes and liquid crystals of salts thereof.

The compounds of the invention include compounds of formula (I) as hereinbefore defined, including all polymorphs and crystal habits thereof, prodrugs and isotopically-labeled compounds of formula (I).

As indicated, so-called ‘prodrugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Prodrugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include
(i) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(ii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of the formula (I).

Also included within the scope of the invention are metabolites of compounds of formula (I), that is, compounds formed in vivo upon administration of the drug. Some examples of metabolites in accordance with the invention include:
(i) where the compound of formula (I) contains a methyl group, an hydroxymethyl derivative thereof (—CH₃->-CH₂OH);
(ii) where the compound of formula (I) contains a phenyl moiety, a phenol derivative thereof (-Ph->-PhOH); and

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N and oxygen, such as ¹⁵O, ¹⁷O and ¹⁸O.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose. Preferably, the compounds according to the present invention are administered as crystalline products.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by a pre-filled capsule, blister or pocket or by a system that uses a gravimetrically fed dosing chamber. Units in accordance with the invention are typically arranged to administer a metered dose or “puff” containing from 1 to 5000 μg of the compound of formula (I) according to the present invention, or a salt thereof. The overall daily dose will typically be in the range 1 μg to 20 mg which may be administered in a single dose or, more usually, as divided doses throughout the day.

The compounds of formula (I) are particularly suitable for an administration by inhalation. According to a preferred embodiment, the compounds according to the present invention are administered through a dry powder inhaler. In this case, the compounds according to the present invention are conveniently formulated with lactose so as to form a dry powder.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 0.001 mg to 5000 mg depending, of course, on the mode of administration. For example, oral administration may require a total daily dose of from 0.1 mg to 1000 mg, while an intravenous dose may only require from 0.001 mg to 100 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The compounds of formula (I) have the ability to interact with muscarinic receptors and thereby have a wide range of therapeutic applications, as described further below, because of the essential role which muscarinic receptors play in the physiology of all mammals.

Therefore, a further aspect of the present invention relates to the compounds of formula (I), or the pharmaceutically acceptable salts thereof, or the pharmaceutically acceptable solvates of said compounds or salts, for use in the treatment of diseases, disorders, and conditions in which muscarinic receptors are involved.

The invention thus also relates to the use of the compounds of formula (I), or the pharmaceutically acceptable salts thereof, or the pharmaceutically acceptable solvates of said compounds or salts, for the manufacture of a medicament useful in the treatment or the prevention of diseases, disorders, and conditions in which the muscarinic receptor is involved.

The invention further relates to a method of treatment of a mammal, including a human being, with a muscarinic receptor antagonist including treating said mammal with an effective amount of a compound of the formula (I), or a pharmaceutically acceptable salt thereof, or a pharmaceutically acceptable solvate of said compound or salt.

More specifically, the present invention also concerns the compounds of formula (I), or the pharmaceutically acceptable salts thereof, or the pharmaceutically acceptable solvates of said compounds or salts, for use in the treatment of diseases, disorders, and conditions selected from the group consisting of:
a chronic or acute bronchoconstriction, chronic bronchitis, small airways obstruction, and emphysema;
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension;
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis;
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis;
acute lung injury; and
a bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis.

The compounds of formula (I), or the pharmaceutically acceptable salts thereof, or the pharmaceutically acceptable solvates of said compounds or salts, are preferably used in the treatment of COPD or asthma.

Other examples of diseases, disorders, and conditions which may be treated by the compounds according to the present invention are inflammatory bowel disease, irritable bowel disease, diverticular disease, motion sickness, gastric ulcers, radiological examination of the bowel, symptomatic treatment of BPH (benign prostatic hyperplasia), NSAID induced gastric ulceration, urinary incontinence (including urgency, frequency, urge incontinence, overactive bladder, nocturia and Lower urinary tract symptoms), cycloplegia, mydriatics and Parkinson's disease.

As COPD and asthma are chronic diseases, compounds used for the therapy will often be co-administered with other drugs. Therefore drug-drug interaction potential can play an important role in the overall safety of a molecule. Compounds which exhibit multiple routes of metabolism i.e. phase I and phase II (for example glucuronidation) have a lower potential for causing a significant drug-drug interaction. Using in vitro metabolism data the potential for drug-drug interaction of a compound can be simulated with commercially available software, for example Simcyp®. Compounds in the current invention exhibit the advantage of both phase I and phase II metabolism resulting in a lower potential for drug-drug interaction compared to the prior art.

Suitable examples of other therapeutic agents which may be used in combination with the compound(s) of formula (I), or the pharmaceutically acceptable salts thereof, or the pharmaceutically acceptable solvates of said compounds or salts, include, but are by no means limited to:
(a) 5-Lipoxygenase (5-LO) inhibitors or 5-lipoxygenase activating protein (FLAP) antagonists;
(b) Leukotriene antagonists (LTRAs) including antagonists of LTB₄, LTC₄, LTD₄, and LTE₄;
(c) Histamine receptor antagonists including H1 and H3 antagonists;
(d) α₁- and α₂-adrenoceptor agonist vasoconstrictor sympathomimetic agents for decongestant use;
(e) PDE inhibitors including PDE3, PDE4 and PDE5 inhibitors;
(f) Beta 2 receptor agonists;
(g) Theophylline;
(h) Sodium cromoglycate;
(i) COX inhibitors both non-selective and selective COX-1 or COX-2 inhibitors (NSAIDs);
(j) Prostaglandin receptor antagonists and inhibitors of prostaglandin synthase;
(k) Oral and inhaled glucocorticosteroids;
(l) Dissociated agonists of the corticoid receptor (DAGR);
(m) Monoclonal antibodies active against endogenous inflammatory entities;
(n) Anti-tumor necrosis factor (anti-TNF-α) agents;
(o) Adhesion molecule inhibitors including VLA-4 antagonists;
(p) Kinin-B₁- and B₂-receptor antagonists;
(q) Immunosuppressive agents including inhibitors of the IgE pathway and cyclosporine;
(r) Inhibitors of matrix metalloproteases (MMPs);
(s) Tachykinin NK₁, NK₂ and NK₃ receptor antagonists;
(t) Protease inhibitors such as elastase inhibitors;
(u) Adenosine A2a receptor agonists and A2b antagonists;
(v) Inhibitors of urokinase;
(w) Compounds that act on dopamine receptors such as D2 agonists;
(x) Modulators of the NFκβ pathway such as IKK inhibitors;
(y) modulators of cytokine signalling pathyways such as p38 MAP kinase, PI3 kinase, JAK kinase, syk kinase, EGFR or MK-2;
(z) Agents that can be classed as mucolytics or anti-tussive;
(aa) Agents, which enhance responses to inhaled corticosteroids;
(bb) Antibiotics and antiviral agents effective against micro-organisms which can colonise the respiratory tract;
(cc) HDAC inhibitors;
(dd) CXCR2 antagonists;
(ee) Integrin antagonists;
(ff) Chemokines;
(gg) Epithelial sodium channel (ENaC) blockers or Epithelial sodium channel (ENaC) inhibitors;
(hh) P2Y2 Agonists and other Nucleotide receptor agonists;
(ii) Inhibitors of thromboxane;
(jj) Inhibitors of PGD₂ synthesis and PGD₂ receptors (DP1 and DP2/CRTH2);
(kk) Niacin; and
(ll) Adhesion factors including VLAM, ICAM, and ELAM.

According to the present invention, the combinations of the compounds of formula (I) with H3 antagonists, β₂ agonists, PDE4 inhibitors, steroids, especially glucocorticosteroids, adenosine A2a receptor agonists, Modulators of cytokine signalling pathyways such as p38 MAP kinase or syk kinase, or Leukotriene antagonists (LTRAs) including antagonists of LTB₄, LTC₄, LTD₄, and LTE₄, are preferred.

According to the present invention, the combinations of the compounds of formula (I) with:
glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, ciclesonide, and mometasone furoate; or
β2 agonists including in particular salbutamol, terbutaline, bambuterol, fenoterol, salmeterol, formoterol, tulobuterol and their salts; are further preferred.

The following examples illustrate the preparation of the compounds of the formula (I):

### Preparations

### Preparation 1

### Biphenyl-2-yl-carbamic acid 1-(9-(bis(tert-butoxycarbonyl))amino-nonyl)-piperidin-4-yl ester

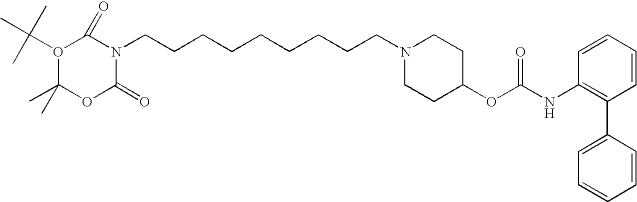

A solution of 4-piperidinyl N-(2-biphenylyl)carbamate hydrochloride (US2006205779, 46.6 g) and N,N-(Bis(tert-butoxycarbonyl)-9-bromononylamine (WO2007107828, 36.1 g) in dimethylformamide (120 ml) and triethylamine (60 ml) was heated at 60° C. for 18 hours under nitrogen. The solvent was removed under reduced pressure and the residue partitioned between ethyl acetate and water (250 ml each). The organic layer was separated, washed with water (100 ml), then with brine (50 ml) and dried over magnesium sulphate. The organic layer was evaporated to dryness to give a brown oil (71.77 g) that was purified by normal phase silica gel column chromatography using heptane:ethyl acetate:880 ammonia as eluant, 60:40:0.5 to 30:70:0.5 (by volume), to give the title compound as a white foam, 38.9 g.

¹H NMR (400 MHz, CDCl₃) δ=1.20-1.25 (m, 10H), 1.30-1.70 (m, 6H), 1.50 (s, 18H), 1.80-1.95 (m, 2H), 2.08-2.23 (m, 2H), 2.50-2.78 (m, 3H), 2.80-2.95 (m, 1H), 3.48-3.54 (m, 2H), 4.78-4.90 (m, 1H), 6.82 (s, 1H), 7.10-7.58 (m, 8H), 8.03-8.16 (d, 1H) ppm.

### Preparation 2

### Biphenyl-2-yl-carbamic acid 1-(9-amino-nonyl)-piperidin-4-yl ester; dihydrochloride salt

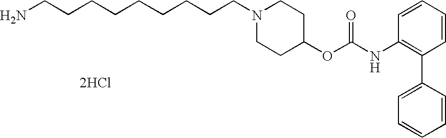

Biphenyl-2-yl-carbamic acid 1-(9-(bis(tert-butoxycarbonyl))amino-nonyl)-piperidin-4-yl ester (Preparation 1, 38.9 g) was taken up in dichloromethane (120 ml), followed by portionwise addition of hydrochloric acid (4M in dioxane, 177 ml) and stirred at room temperature for 18 hours. The solvent and excess acid were removed in vacuo and the residue azeotroped with methanol several times to give the title compound as a colourless powder, 31.98 g.

¹H NMR (CDCl₃, 400 MHz) δ=1.25-1.43 (m, 10H), 1.75-1.89 (m, 4H), 1.99-2.08 (m, 1H), 2.13-2.17 (m, 2H), 2.24-2.33 (m, 1H), 2.48-2.52 (m, 2H), 2.82-2.91 (m, 2H), 2.93-3.06 (m, 2H), 3.48-3.52 (m, 1H), 3.63-3.67 (m, 1H), 4.77-4.85 (m, 1H), 5.06 (s, 1H), 6.77 (d, 1H), 7.11-7.24 (m, 3H), 7.31-7.51 (m, 5H), 8.00 (d, 1H), 8.31 (br s, 1H) ppm.

### Preparation 2a

### Biphenyl-2-yl-carbamic acid 1-(9-amino-nonyl)-piperidin-4-yl ester

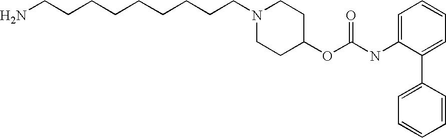

Biphenyl-2-yl-carbamic acid 1-(9-amino-nonyl)-piperidin-4-yl ester dihydrochloride salt (Preparation 2, 5 g, 9.8 mmol) was dissolved in dichloromethane and washed with 1N aqueous sodium hydroxide solution. The organic layer was separated, dried over magnesium sulphate and concentrated in vacuo to yield the title compound, 3.11 g.

¹H NMR (CDCl₃, 400 MHz) δ=1.26-1.30 (m, 10H), 1.38-1.48 (m, 4H), 1.64-1.73 (m, 2H), 1.91-1.97 (m, 2H), 2.15-2.20 (m, 2H), 2.27-2.31 (m, 2H), 2.65-2.69 (t, 2H), 2.70-2.74 (m, 2H), 4.70-4.75 (m, 1H), 6.59 (s, 1H), 7.11-7.14 (m, 1H), 7.21 (d, 1H), 7.33-7.51 (m, 7H), 8.10 (d, 1H) ppm.

### Preparation 3

### 9-Methylamino-nonan-1-ol

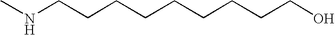

To 9-bromononanol (25 g) was added methylamine (33% solution in ethanol, 200 ml) and the solution stirred for 18 hours at room temperature under nitrogen. The solvent was removed under vacuum, the resulting colourless solid was dissolved in dichloromethane (200 mL), washed with aqueous sodium hydroxide solution (2M, 100 ml), water (100 ml), dried (sodium sulphate) and concentrated in vacuo to give the title compound as a yellow oil that solidified on standing, 14.95 g. Said compound was used as such in preparation 4.

### Preparation 4

### (9-Hydroxy-nonyl)-methyl-carbamic acid tert-butyl ester

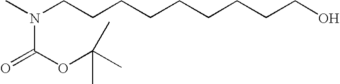

9-Methylamino-nonan-1-ol (Preparation 3, 14.95 g) was suspended in a mixture of dichloromethane (250 ml) and triethylamine (17.6 g) and cooled in an ice-bath with stirring. Boc anhydride (18.8 g) was added in portions over 5 minutes and the reaction stirred in the ice bath for 1 hour and then at room temperature for 4 hours. The reaction was washed with water (150 ml), 10% aqueous citric acid solution (50 ml) and saturated brine (50 ml), then dried (sodium sulphate) and concentrated in vacuo to give the title compound as a yellow liquid, 22.95 g, 97%.

¹H NMR (400 MHz, CDCl₃) δ=1.20-1.38 (m, 10H), 1.47 (s, 9H), 1.47-1.60 (m, 4H), 2.80 (s, 3H), 3.10-3.22 (t, 2H), 3.78-3.83 (t, 2H) ppm.

### Preparation 5

### Methanesulfonic acid 9-(tert-butoxycarbonyl-methyl-amino)-nonyl ester

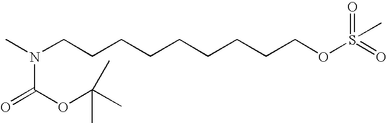

To a solution of (9-hydroxy-nonyl)-methyl-carbamic acid tert-butyl ester (Preparation 4, 22.95 g) in dichloromethane (230 mL) and triethylamine (18 mL) at 5° C., was added methane sulphonyl chloride (7.2 mL) dropwise and the viscous, cloudy solution stirred at room temperature for 1 hour. The mixture was washed with water, saturated aqueous sodium bicarbonate solution and the organic layer dried (magnesium sulphate) and evaporated in vacuo to give the title compound as a light yellow oil, 29.30 g.

¹H NMR (400 MHz, CDCl₃) δ=1.20-1.45 (m, 10H), 1.44 (s, 9H), 1.44-1.52 (m, 2H), 1.70-1.79 (m, 2H), 2.82 (s, 3H), 2.98 (s, 3H), 3.14-3.24 (m, 2H), 4.20-4.4.24 (t, 2H) ppm.

### Preparation 6

### Biphenyl-2-yl-carbamic acid 1-[9-(tert-butoxycarbonyl-methyl-amino)-nonyl]-piperidin-4-yl ester

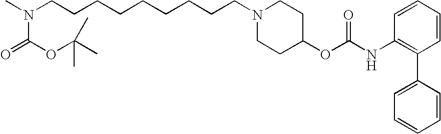

4-Piperidinyl-N-(2-biphenylyl)carbamate hydrochloride (US2006205779, 29.3 g) was stirred with potassium carbonate (46 g) in dimethylformamide (250 ml) for 0.5 hour. Methanesulfonic acid 9-(tert-butoxycarbonyl-methyl-amino)-nonyl ester (Preparation 5, 27.7 g) and potassium iodide (277 mg) were then added. The reaction mixture was stirred at 65° C. for 24 hours, then additional dimethylformamide (100 mL) was added to aid stirring at 65° C. for a further 24 hours. The solvent was removed in vacuo and the residue partitioned between water and ethyl acetate (500 ml each). The aqueous layer was separated and extracted with further ethyl acetate (200 ml). The combined organic layers were washed with saturated brine, dried (sodium sulphate) and concentrated in vacuo. The crude residue (46.46 g) was purified by normal phase silica gel column chromatography using ethyl acetate:heptane:880 ammonia (80:20:0.5, by volume) as eluant to give the title compound as a colourless oil which crystallised on standing, 30 g, 65%.

¹H NMR (400 MHz, CDCl₃) δ=1.22-1.38 (m, 12H), 1.44 (s, 9H), 1.44-1.56 (m, 2H), 1.61-1.73 (m, 2H), 1.88-1.97 (m, 2H), 2.12-2.24 (t, 2H), 2.23-2.30 (t, 2H), 2.64-2.72 (m, 2H), 2.82 (s, 3H), 3.16-3.24 (m, 2H), 4.63-4.78 (m, 1H), 6.60 (s, 1H), 7.08-7.56 (m, 8H), 8.03-8.15 (d, 1H) ppm.

### Preparation 7

### Biphenyl-2-yl-carbamic acid 1-(9-methylamino-nonyl)-piperidin-4-yl ester; dihydrochloride salt

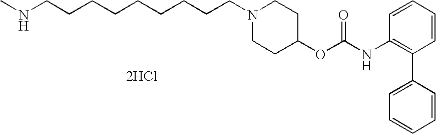

Biphenyl-2-yl-carbamic acid 1-[9-(tert-butoxycarbonyl-methyl-amino)-nonyl]-piperidin-4-yl ester (Preparation 6, 18.5 g) was stirred in a solution of hydrochloric acid in dioxane (85 ml, 4M) at room temperature for 18 hours. The solvent and excess acid were removed in vacuo and the residue azeotroped twice with dichloromethane (100 ml) to give the title compound as a white solid, 18.0 g.

¹H NMR (400 MHz, CDCl₃) δ=1.22-1.38 (m, 10H), 1.54-2.10 (m, 8H), 2.78-2.97 (m, 4H), 3.29-3.42 (m, 2H), 3.53-3.65 (m, 2H), 3.57 (s, 3H), 4.57-4.67 (m, 1H), 4.74 (bs, 1H), 7.30-7.45 (m, 8H), 8.80-8.90 (m, 3H) 10.71-10.87 (m, 1H) ppm.

### Preparation 7a

### Biphenyl-2-yl-carbamic acid 1-(9-methylamino-nonyl)-piperidin-4-yl ester

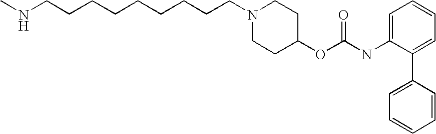

Biphenyl-2-yl-carbamic acid 1-(9-methylamino-nonyl)-piperidin-4-yl ester; dihydrochloride salt (Preparation 7, 1.355 g, 2.583 mmol) was dissolved in water (20 ml) and treated with 1N aqueous sodium hydroxide solution (6 ml, 6 mmol). The resulting white suspension was extracted with dichloromethane (40 ml), and then with dichloromethane:methanol (40 ml, 95:5 by volume). The combined organic layers were dried (magnesium sulphate) and concentrated in vacuo to yield the title compound as a colourless glass which crystallised on standing, in 96% yield, 1.116 g.

¹H NMR (CDCl₃, 400 MHz): δ=1.25-1.34 (m, 10H), 1.45-1.51 (m, 2H), 1.67-1.80 (m, 4H), 1.95-1.99 (m, 2H), 2.23-2.28 (m, 2H), 2.32-2.36 (t, 2H), 2.61 (s, 3H), 2.71-2.76 (m, 2H), 2.81-2.85 (t, 2H), 4.70-4.75 (m, 1H), 6.58 (s, 1H), 7.10-7.14 (t, 1H), 7.21 (d, 1H), 7.33-7.50 (m, 6H), 8.08 (d, 1H) ppm.

### Preparation 8

### 4-Chloro-5-fluoro-2-hydroxy-benzaldehyde

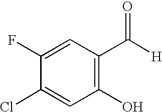

Hexamethylenetetramine (210 g, 1.5 mol) was added to trifluoroacetic acid (3.6 L) in small portions and the resulting mixture was heated to reflux at 78° C. A solution of 3-chloro-4-fluorophenol (210 g, 1.43 mol) in trifluoroacetic acid (1.4 L) was then added dropwise and the mixture stirred for another 1 hour. The mixture was cooled to room temperature, and concentrated in vacuo. The residue was poured into ice-water (2 L) and stirred overnight. The mixture was filtered and the filter cake dissolved in ethyl acetate (500 mL), dried over magnesium sulfate and concentrated in vacuo. This crude residue was washed with ethyl acetate/petroleum ether (10:1, by volume) to give the title compound as a white solid, 56.5 g, 23%.

¹H NMR (400 MHz, CDCl₃) δ=7.00-7.01 (m, 1H), 7.19-7.26 (m, 1H), 9.75 (s, 1H), 10.82 (s, 1H) ppm.

LCMS: m/z 172.9 M−

### Preparation 9

### (2,6-Dichloro-phenoxy)-triethyl-silane

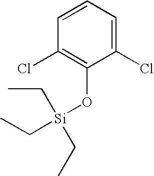

2,6-dichlorophenol (16.3 g, 100 mmol) was dissolved in tetrahydrofuran (300 ml). To this solution was added anhydrous pyridine (16.2 ml, 200 mmol) and chlorotriethylsilane (22.7 ml, 135 mmol). The resulting reaction mixture was stirred at room temperature for 18 hours, and then at 80° C. for 5 hours. The reaction was cooled to room temperature and poured onto saturated aqueous sodium hydrogen carbonate solution (150 ml) and extracted with dichloromethane (3×80 ml). The combined organic layers were dried (sodium sulphate) and concentrated in vacuo to yield crude product. The residue was purified by column chromatography on silica gel eluting with heptane to furnish the title compound as a colourless oil, in 54% yield, 15 g.

¹H NMR (400 MHz, CDCl₃) δ=0.83-0.89 (q, 6H), 0.99-1.03 (t, 9H), 6.80-6.84 (m, 1H), 7.23-7.26 (m, 2H) ppm.

### Preparation 10

### 2,4-Dichloro-3-hydroxy-benzaldehyde

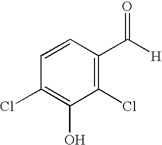

Sec-butyllithium in cyclohexane (1.4M, 34.5 ml, 48.3 mmol) was added slowly to a solution of (2,6-dichloro-phenoxy)-triethyl-silane (Preparation 7, 12.17 g, 43.89 mmol) in tetrahydrofuran (100 ml) at −72° C. When addition was complete, the reaction was stirred at −72° C. for 1 hour. Anhydrous dimethylformamide (4.42 ml, 57.1 mmol) was then added, keeping the temperature of the reaction below −65° C. Stirring was continued at −65° C. for 10 minutes and then at −65° C. to room temperature over 0.5 hours. The reaction was quenched by the addition of 2N hydrochloric acid solution saturated with sodium chloride (100 ml) and the resulting mixture extracted with ethyl acetate (100 ml). The organic layer was washed with brine (100 ml), dried (sodium sulphate) and concentrated in vacuo to yield the crude product. Trituration in heptane:dichloromethane (10:1, by volume; 220 ml) yielded the title compound as a white solid, in 65% yield, 5.5 g.

LCMS: m/z 188 M−

¹H NMR (400 MHz, DMSO-d₆) δ 7.32 (d, 1H), 7.49 (d, 1H), 10.23 (s, 1H) ppm.

### EXAMPLES

### Example 1

### Biphenyl-2-yl-carbamic acid 1-[9-(3-chloro-4-hydroxy-benzoylamino)-nonyl]-piperidin-4-yl ester

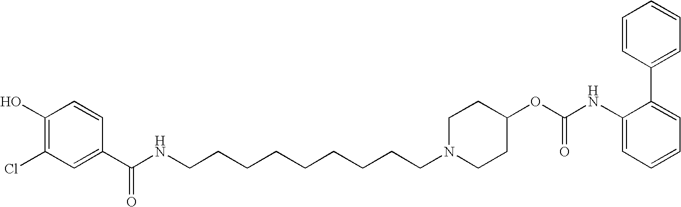

To a solution of biphenyl-2-yl-carbamic acid 1-(9-amino-nonyl)-piperidin-4-yl ester dihydrochloride salt (Preparation 2, 200 mg, 457 μmol) in dichloromethane (5 ml), were added triethylamine (191 μl, 1.37 mmol), 3-chloro-4-hydroxybenzoic acid (91.3 mg, 503 μmol), 1-hydroxy benzotriazole monohydrate (84 mg, 548 μmol) and (3-(dimethylamino)propyl)ethyl carbodiimide hydrochloride (105 mg, 548 μmol) and the mixture was stirred at room temperature for 72 hours. The resulting clear solution was washed with saturated aqueous sodium hydrogen carbonate solution (5 ml), water (5 ml), brine (5 ml), dried (sodium sulphate) and the solvent removed in vacuo to yield a colourless gum. The residue was purified using a RediSep® silica gel cartridge eluting with dichloromethane:methanol:0.88 ammonia (99:1:0.1 to 90:10:1, by volume) to afford the title compound as a white foam, 73% yield, 198 mg.

¹H NMR (400 MHz, METHANOL-d₄) δ=1.27-1.40 (m, 10H), 1.46-1.55 (m, 2H), 1.56-1.68 (m, 4H), 1.82-1.90 (m, 2H), 2.32-2.42 (m, 4H), 2.66-2.76 (m, 2H), 3.31-3.35 (t, 2H), 4.58-4.66 (m, 1H), 6.92 (d, J=8.19 Hz, 1H), 7.23-7.45 (m, 8H), 7.52-7.56 (m, 1H), 7.60 (dd, J=8.39, 2.15 Hz, 1H), 7.80 (d, 1H) ppm.

LCMS: m/z 592 [M+H]+, 590 M−

### Example 2

### Biphenyl-2-yl-carbamic acid 1-[9-(2-fluoro-3-hydroxy-benzylamino)-nonyl]-piperidin-4-yl ester

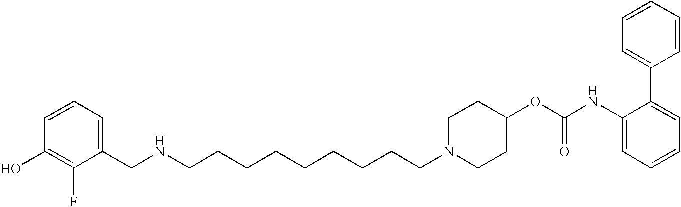

To a solution of biphenyl-2-yl-carbamic acid 1-(9-amino-nonyl)-piperidin-4-yl ester (Preparation 2, 150 mg, 343 μmol) in ethanol (5 ml) were added 2-fluoro-3-hydroxybenzaldehyde (Synthesis, (9), 710-12, 1988; 48.1 mg, 343 μmol), acetic acid (in excess of 0.02 ml, 343 μmol) and sodium sulfate (drying agent), and the mixture was stirred under nitrogen at room temperature for 1 hour. Sodium triacetoxyborohydride was then added (145 mg, 686 μmol) and the mixture stirred under nitrogen at room temperature for 24 hours. The mixture was diluted with water (2 ml), the solvent removed in vacuo and the residue partitioned between saturated sodium hydrogen carbonate solution (10 ml) and dichloromethane (10 ml). The organic layer was separated, washed with brine (5 ml), dried (sodium sulphate) and the solvent removed in vacuo to yield a colourless gum. The residue was purified using a RediSep® silica gel cartridge eluting with dichloromethane:methanol:0.88 ammonia (99:1:0.1 to 95:5:0.5, by volume) to afford the title compound as a colourless gum, 47% yield, 90 mg.

¹H NMR (400 MHz, METHANOL-d₄) δ=1.27-1.36 (m, 10H), 1.45-1.56 (m, 4H), 1.56-1.66 (m, 2H), 1.81-1.89 (m, 2H), 2.23-2.35 (m, 4H), 2.58-2.71 (m, 4H), 3.80 (s, 2H) 4.56-4.63 (m, 1H), 6.75-6.78 (td, 1H), 6.80-6.85 (td, 1H), 6.91-6.95 (td, 1H), 7.23-7.44 (m, 8H), 7.52-7.56 (m, 1H) ppm.

LRMS: m/z 562 [M+H]+, 560 M−

### Example 3

### Biphenyl-2-yl-carbamic acid 1-[9-(3-chloro-5-fluoro-2-hydroxy-benzylamino)-nonyl]-piperidin-4-yl ester

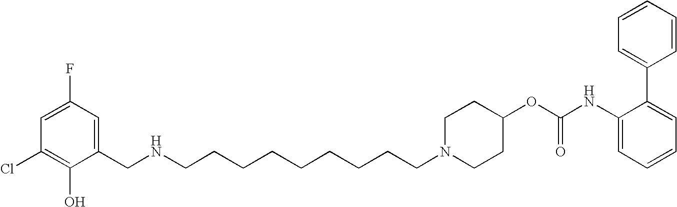

The title compound was prepared from biphenyl-2-yl-carbamic acid 1-(9-amino-nonyl)-piperidin-4-yl ester (Preparation 2, 130 mg, 297 μmol) and 3-chloro-5-fluoro-2-hydroxybenzaldehyde (51.8 mg, 297 μmol) using the same method as described in example 2. Additional crystallisation of the product in methanol afforded the title compound as a crystalline white solid, 43% yield, 77 mg.

¹H NMR (400 MHz, METHANOL-d₄) δ=1.26-1.41 (m, 10H), 1.45-1.53 (m, 2H), 1.57-1.67 (m, 4H), 1.80-1.89 (m, 2H), 2.23-2.35 (m, 4H), 2.62-2.70 (m, 2H), 2.74-2.78 (t, 2H), 4.00 (s, 2H), 4.56-4.63 (m, 1H), 6.76-6.79 (dd, 1H), 6.99-7.02 (dd, 1H), 7.23-7.44 (m, 8H), 7.52-7.56 (m, 1H) ppm.

LRMS: m/z 596 [M+H]+, 594 M−

### Example 4

### Biphenyl-2-yl-carbamic acid 1-{9-[2-(3-chloro-4-hydroxy-phenyl)-acetylamino]-nonyl}-piperidin-4-yl ester

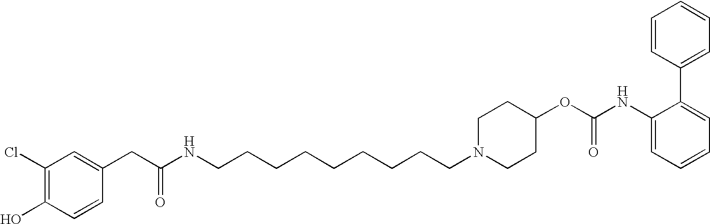

The title compound was prepared from biphenyl-2-yl-carbamic acid 1-(9-amino-nonyl)-piperidin-4-yl ester dihydrochloride salt (Preparation 2, 100 mg, 229 μmol) and 3-chloro-4-hydroxyphenylacetic acid (46.9 mg, 251 μmol) using the same method as described in example 1 to afford the title compound as a white foam, 72% yield, 100 mg.

¹H NMR (400 MHz, METHANOL-d₄) δ=1.27-1.35 (m, 10H), 1.46-1.55 (m, 4H), 1.58-1.68 (m, 2H), 1.82-1.90 (m, 2H), 2.28-2.37 (m, 4H), 2.66-2.74 (m, 2H), 3.15-3.19 (t, 2H), 3.35 (s, 2H), 4.58-4.66 (m, 1H), 6.82-6.84 (d, 1H), 7.01-7.04 (1H, dd), 7.23-7.45 (m, 9H), 7.52-7.56 (m, 1H) ppm.

### LCMS: m/z 606 [M+H]+

### Example 5

### Biphenyl-2-yl-carbamic acid 1-[9-(2-chloro-3-hydroxy-benzylamino)-nonyl]-piperidin-4-yl ester

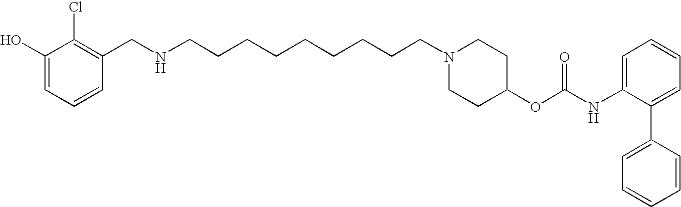

The title compound was prepared from biphenyl-2-yl-carbamic acid 1-(9-amino-nonyl)-piperidin-4-yl ester (Preparation 2, 130 mg, 297 μmol) and 2-chloro-3-hydroxybenzaldehyde (46.5 mg, 297 μmol) using the same method as described in example 2 to afford the title compound as a colourless glass, 39% yield, 67 mg.

¹H NMR (400 MHz, METHANOL-d₄) δ=1.26-1.381 (m, 10H), 1.45-1.68 (m, 6H), 1.80-1.89 (m, 2H), 2.23-2.35 (m, 4H), 2.60-2.72 (m, 4H), 3.88 (s, 2H), 4.56-4.63 (m, 1H), 6.85-6.89 (2×d, 2H), 7.08-7.12 (dd, 1H), 7.23-7.44 (m, 8H), 7.52-7.56 (m, 1H) ppm.

LRMS: m/z 578-80 [M+H]+, 576-578 M−

### Example 6

### Biphenyl-2-yl-carbamic acid 1-[9-(3-fluoro-4-hydroxy-benzoylamino)-nonyl]-piperidin-4-yl ester

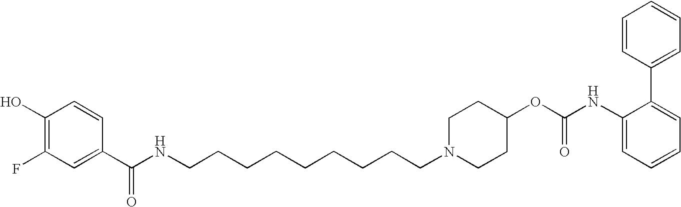

The title compound was prepared from biphenyl-2-yl-carbamic acid 1-(9-amino-nonyl)-piperidin-4-yl ester dihydrochloride salt (Preparation 2, 2.20 g, 4.31 mmol) and 3-fluoro-4-hydroxybenzoic acid (740 mg, 4.74 mmol) using the same method as described in example 1 to afford the title compound as a white foam, 51% yield, 1.26 g.

¹H NMR (400 MHz, METHANOL-d₄) δ=1.27-1.41 (m, 10H), 1.46-1.54 (m, 2H), 1.55-1.68 (m, 4H), 1.81-1.89 (m, 2H), 2.30-2.42 (m, 4H), 2.65-2.75 (m, 2H), 3.31-3.35 (t, 2H), 4.58-4.65 (m, 1H), 6.90-6.95 (dd, 1H), 7.23-7.56 (m, 11H) ppm.

LCMS: m/z 576 [M+H]+, 574 M−

### Example 7

### Biphenyl-2-yl-carbamic acid 1-[9-(4-fluoro-3-hydroxy-benzoylamino)-nonyl]-piperidin-4-yl ester

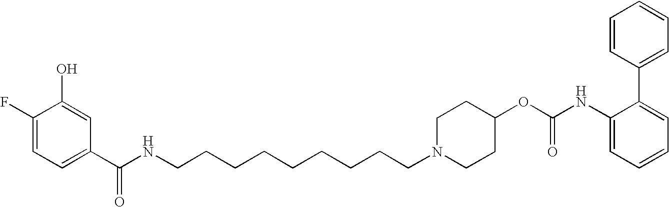

The title compound was prepared from biphenyl-2-yl-carbamic acid 1-(9-amino-nonyl)-piperidin-4-yl ester dihydrochloride salt (Preparation 2, 30.0 mg, 68.6 μmol) and 4-fluoro-3-hydroxybenzoic acid (11.8 mg, 75.4 μmol) using the same method as described in example 1 with stirring for 5 days. Aqueous sodium hydrogen carbonate solution (5 ml) was added, the mixture stirred for 2 hours and filtered through a phase separation cartridge. The organic layer was reduced in vacuo and the crude material was purified by HPLC method D to afford the title compound.

LCMS Method D RT 2.53 min (100% area)

ES m/z 576.316 [M+H]⁺

### Example 8

### Biphenyl-2-yl-carbamic acid 1-[9-(3-fluoro-2-hydroxy-benzoylamino)-nonyl]-piperidin-4-yl ester

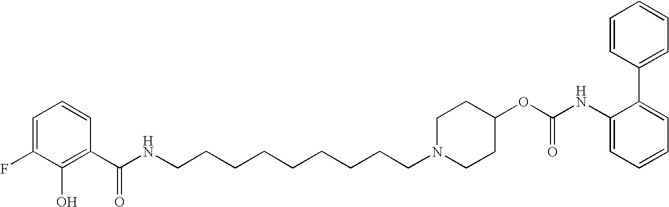

The title compound was prepared from biphenyl-2-yl-carbamic acid 1-(9-amino-nonyl)-piperidin-4-yl ester dihydrochloride salt (Preparation 2, 30.0 mg, 68.6 μmol) and 3-fluoro-2-hydroxybenzoic acid (11.8 mg, 75.4 μmol) using the same method as described in example 7.

LCMS Method D: RT 2.69 min (100% area)

ES m/z 576.316 [M+H]⁺

### Example 9

### Biphenyl-2-yl-carbamic acid 1-[9-(5-fluoro-2-hydroxy-benzoylamino)-nonyl]-piperidin-4-yl ester

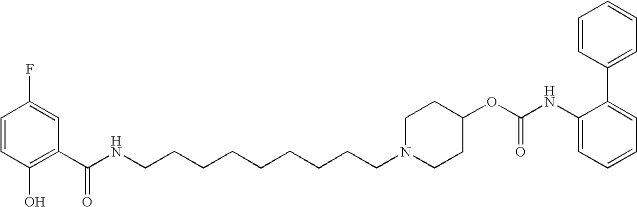

The title compound was prepared from biphenyl-2-yl-carbamic acid 1-(9-amino-nonyl)-piperidin-4-yl ester dihydrochloride salt (Preparation 2, 30.0 mg, 68.6 μmol) and 5-fluoro-2-hydroxybenzoic acid (11.8 mg, 75.4 μmol) using the same method as described in example 7.

LCMS Method D: RT 2.69 min (100% area)

ES m/z 576.316 [M+H]⁺

### Example 10

### Biphenyl-2-yl-carbamic acid 1-{9-[2-(3-fluoro-4-hydroxy-phenyl)-acetylamino]-nonyl}-piperidin-4-yl ester

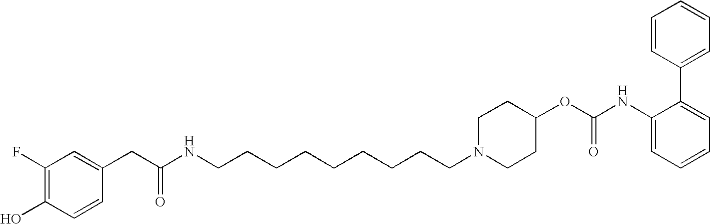

The title compound was prepared from biphenyl-2-yl-carbamic acid 1-(9-amino-nonyl)-piperidin-4-yl ester dihydrochloride salt (Preparation 2, 30.0 mg, 68.6 μmol) and 3-fluoro-4-hydroxyphenylacetic acid (12.8 mg, 75.4 μmol) using the same method as described in example 7.

LCMS Method D: RT 2.32 min (100% area)

ES m/z 590.332 [M+H]⁺

### Example 11

### Biphenyl-2-yl-carbamic acid 1-[9-(3-chloro-2-hydroxy-benzoylamino)-nonyl]-piperidin-4-yl ester

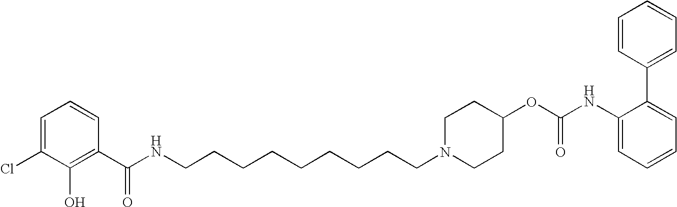

The title compound was prepared from biphenyl-2-yl-carbamic acid 1-(9-amino-nonyl)-piperidin-4-yl ester dihydrochloride salt (Preparation 2, 30.0 mg, 68.6 μmol) and 3-chloro-2-hydroxybenzoic acid (13.0 mg, 75.4 μmol) using the same method as described in example 7.

LCMS Method D: RT 2.68 min (100% area)

ES m/z 592.28 [M+H]⁺

### Example 12

### Biphenyl-2-yl-carbamic acid 1-[9-(3,4-difluoro-2-hydroxy-benzoylamino)-nonyl]-piperidin-4-yl ester

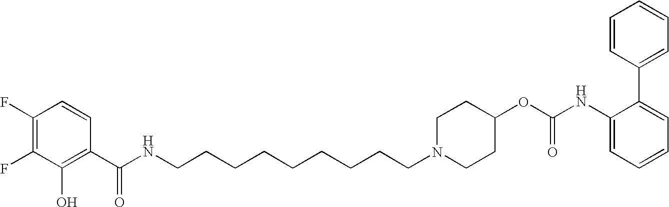

The title compound was prepared from biphenyl-2-yl-carbamic acid 1-(9-amino-nonyl)-piperidin-4-yl ester dihydrochloride salt (Preparation 2, 30.0 mg, 68.6 μmol) and 3,4-difluoro-2-hydroxybenzoic acid (13.1 mg, 75.4 μmol) using the same method as described in example 7.

LCMS Method D: RT 2.61 min (100% area)

ES m/z 594.307 [M+H]⁺

### Example 13

### Biphenyl-2-yl-carbamic acid 1-[9-(3,5-dichloro-2-hydroxy-benzoylamino)-nonyl]-piperidin-4-yl ester

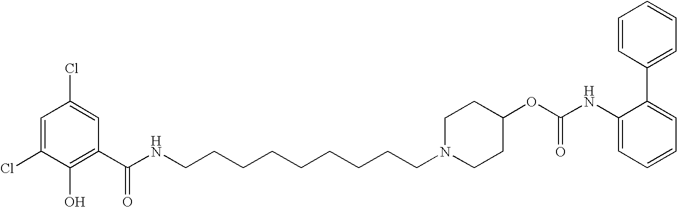

The title compound was prepared from biphenyl-2-yl-carbamic acid 1-(9-amino-nonyl)-piperidin-4-yl ester dihydrochloride salt (Preparation 2, 30.0 mg, 68.6 μmol) and 3,5-dichloro-2-hydroxybenzoic acid (15.6 mg, 75.4 μmol) using the same method as described in example 7.

LCMS Method D: RT 2.91 min (100% area)

ES m/z 626.247 [M+H]⁺

### Example 14

### Biphenyl-2-yl-carbamic acid 1-[9-(4-chloro-2-hydroxy-benzoylamino)-nonyl]-piperidin-4-yl ester

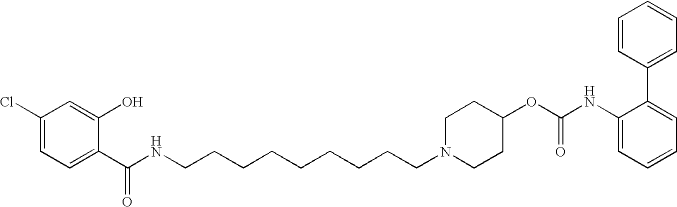

The title compound was prepared from biphenyl-2-yl-carbamic acid 1-(9-amino-nonyl)-piperidin-4-yl ester dihydrochloride salt (Preparation 2, 45.0 mg, 88 μmol) and 4-chloro-2-hydroxybenzoic acid (15.2 mg, 88 μmol) using the same method as described in example 7.

LCMS Method D: RT 2.62 min (100% area) ES m/z 592.28 [M+H]⁺

### Example 15

### Biphenyl-2-yl-carbamic acid 1-(9-{[2-(3-chloro-4-hydroxy-phenyl)-acetyl]-methyl-amino}-nonyl)-piperidin-4-yl ester

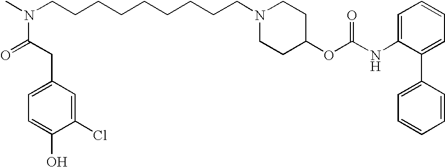

To a solution of biphenyl-2-yl-carbamic acid 1-(9-methylamino-nonyl)-piperidin-4-yl ester dihydrochloride salt (Preparation 7, 20.0 mg, 44 μmol) in dichloromethane (2 ml), was added triethylamine (9.26 μl, 66 μmol), 3-chloro-4-hydroxyphenylacetic acid (8.26 mg, 44 μmol) and O-(1H-benzotriazol-1-yl)-N,N,N′,N′-tetramethyluronium hexafluoro-phosphate (21.8 mg, 58 μmol) and stirred at room temperature for 5 days. The solvent was removed in vacuo and the crude residue purified using an Isolute® SCX-1 cartridge, eluting with methanol followed by 2M ammonia in methanol. The basic fractions were evaporated under reduced pressure and the residue purified by HPLC method F to afford the title compound.

LCMS Method F: RT 2.7 min (100% area)

ES m/z 620.318 [M+H]⁺

Alternatively, the title compound was also prepared by the following procedure:

To a solution of biphenyl-2-yl-carbamic acid 1-(9-methylamino-nonyl)-piperidin-4-yl ester dihydrochloride salt (Preparation 7, 3.0 g, 5.72 mmol) in dimethylformamide (30 ml), was added triethylamine (1.63 ml, 11.7 mmol), 3-chloro-4-hydroxyphenylacetic acid (1.28 g, 6.86 mmol), N,N-dimethylaminopyridine (210 mg, 1.72 mmol) and (3-(dimethylamino)propyl)ethyl carbodiimide hydrochloride (1.32 g, 6.86 mmol). The reaction was stirred at room temperature for 18 hours. The solvent was removed in vacuo and residue partitioned between ethyl acetate (50 ml) and water (50 ml). The organic layer was washed with saturated aqueous sodium bicarbonate solution (2×50 ml), dried over magnesium sulphate and concentrated in vacuo. The residue was purified by column chromatography on silica gel eluting with dichloromethane:methanol:880 ammonia (96:4:0.4 to 92:8:0.8, by volume), to furnish the title compound as a white foam, in 50% yield, 1.77 g.

Solubilisation of 50 mg of the title compound in either isopropyl alcohol, isopropyl acetate or ethyl acetate (1 ml, hot), followed by standing for 72 hours at room temperature and filtration afforded the title compound as a crystalline solid.

LCMS: APCI ESI m/z 621 [M+H]⁺

¹H NMR (400 MHz, METHANOL-d₄) δ=1.17-1.34 (m, 10H), 1.37-1.55 (m, 2H), 1.59-1.67 (m, 4H), 1.80-1.89 (m, 2H), 2.24-2.35 (m, 4H), 2.62-2.72 (m, 2H), 2.90-3.00 (d, 3H), 3.34-3.39 (m, 2H), 3.62-3.65 (d, 2H), 4.57-4.63 (m, 1H), 6.84 (d, 1H), 6.99 (d, 1H), 7.18-7.44 (m, 9H), 7.56 (d, 1H) ppm.

### Example 16

### Biphenyl-2-yl-carbamic acid 1-{9-[(4-fluoro-3-hydroxy-benzoyl)-methyl-amino]-nonyl}-piperidin-4-yl ester

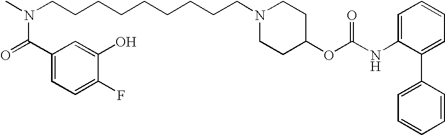

The title compound was prepared from biphenyl-2-yl-carbamic acid 1-(9-methylamino-nonyl)-piperidin-4-yl ester dihydrochloride salt (Preparation 7, 20.0 mg, 44 μmol) and 4-fluoro-3-hydroxybenzoic acid (6.91 mg, 44 μmol) using the same method as described in example 15.

LCMS Method F: RT 2.59 min (100% area)

ES m/z 590.332 [M+H]⁺

### Example 17

### Biphenyl-2-yl-carbamic acid 1-{9-[(3-chloro-4-hydroxy-benzoyl)-methyl-amino]-nonyl}-piperidin-4-yl ester

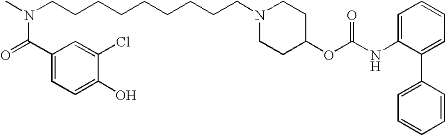

The title compound was prepared from biphenyl-2-yl-carbamic acid 1-(9-methylamino-nonyl)-piperidin-4-yl ester dihydrochloride salt (Preparation 7, 20.0 mg, 44 μmol) and 3-chloro-4-hydroxybenzoic acid (7.64 mg, 44 μmol) using the same method as described in example 15.

LCMS Method F: RT 2.67 min (100% area)

ES m/z 606.302 [M+H]⁺

Alternatively, the title compound was also prepared by the following procedure:

To a solution of biphenyl-2-yl-carbamic acid 1-(9-methylamino-nonyl)-piperidin-4-yl ester dihydrochloride salt (Preparation 7, 4.0 g, 8.856 mmol) in tetrahydrofuran (100 ml), was added 3-chloro-4-hydroxybenzoic acid (1.69 g, 9.30 mmol), N,N-dimethylaminopyridine (433 mg, 3.54 mmol) and (3-(dimethylamino)propyl)ethyl carbodiimide hydrochloride (2.04 g, 10.6 mmol). The reaction was stirred at room temperature for 18 hours. The mixture was partitioned between ethyl acetate (75 ml) and water (75 ml). The aqueous layer was further extracted with ethyl acetate (75 ml) and the combined organic layers washed with brine (75 ml), dried over magnesium sulphate and concentrated in vacuo. The residue was dissolved in methanol/water (100 ml/20 ml) and treated with potassium carbonate (9.8 g, 70.9 mmol) and heated at 50° C. for 3 hours. The solvent was removed in vacuo and residue partitioned between ethyl acetate (75 ml) and water (75 ml). The aqueous layer was adjusted to pH8 by addition of aqueous hydrochloric acid, and further extracted with ethyl acetate (75 ml). Combined organic extracts washed with brine (50 ml), dried over magnesium sulphate and concentrated in vacuo. The residue was purified by column chromatography on silica gel eluting with ethyl acetate:methanol:880 ammonia (100:0:0 to 90:10:1, by volume), to furnish the title compound as a white foam, in 25% yield, 1.1 g.

150 mg of title compound was suspended in acetonitrile (4 ml), heated to reflux and then allowed to cool to room temperature. The resulting oil/solvent mixture was heated to 80° C. until crystallisation had occurred and then allowed to cool to room temperature. The solid was collected by filtration to yield the title compound as a white crystalline solid, 102 mg.

LCMS: APCI ESI m/z 606 [M+H]⁺

¹H NMR (400 MHz, DMSO-d₆) δ=1.11-1.23 (m, 10H), 1.30-1.44 (m, 4H), 1.46-1.54 (m, 2H), 1.65-1.73 (m, 2H), 1.99-2.07 (m, 2H), 2.15-2.21 (m, 2H), 2.51-2.58 (m, 2H), 2.88 (s, 3H), 3.15-3.19 (m, 2H), 4.37-4.45 (m, 1H), 6.95 (d, 1H), 7.15 (d, 1H), 7.24-7.42 (m, 9H), 8.54 (d, 1H) ppm.

### Example 18

### Biphenyl-2-yl-carbamic acid 1-(9-{[2-(3-fluoro-4-hydroxy-phenyl)-acetyl]-methyl-amino}-nonyl)-piperidin-4-yl ester

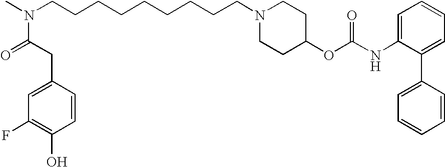

The title compound was prepared from biphenyl-2-yl-carbamic acid 1-(9-methylamino-nonyl)-piperidin-4-yl ester dihydrochloride salt (Preparation 7, 20.0 mg, 44 μmol) and 3-fluoro-4-hydroxyphenylacetic acid (8.29 mg, 49 μmol) using the same method as described in example 15.

LCMS Method F: RT 2.6 min (100% area)

ES m/z 604.347 [M+H]⁺

### Example 19

### Biphenyl-2-yl-carbamic acid 1-[9-(4-chloro-3-hydroxy-benzoylamino)-nonyl]-piperidin-4-yl ester

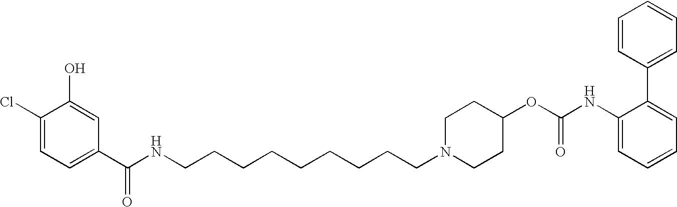

(3-(Dimethylamino)propyl)ethylcarbodimide hydrochloride (69.6 mg, 0.363 mmol) was added to a solution of biphenyl-2-yl-carbamic acid 1-(9-amino-nonyl)-piperidin-4-yl ester (Preparation 2, 159 mg, 0.363 mmol), 4-chloro-3-hydroxybenzoic acid (56.4 mg, 0.327 mmol) and 1-hydroxybenzotriazole monohydrate (55.6 mg, 0.363 mmol) in a mixture of dichloromethane (2 ml) and dimethylformamide (1 ml) and stirred for 24 hours at room temperature under nitrogen. The solvent was removed in vacuo and the residue was partitioned between dichloromethane (40 ml) and saturated sodium hydrogen carbonate solution (30 ml). The layers were separated and the aqueous layer extracted with further dichloromethane (40 ml). The combined organic layers were dried (magnesium sulphate), the solvent removed in vacuo and the crude residue purified by column chromatography on silica gel eluting with ethyl acetate:methanol:880 ammonia, 98:2:0.2 to 90:10:1 (by volume), to furnish the title compound as a white foam, 63% yield, 135 mg.

¹H NMR (400 MHz, METHANOL-d₄) δ=1.26-1.41 (m, 10H), 1.47-1.54 (m, 2H), 1.56-1.66 (m, 4H), 1.80-1.89 (m, 2H), 2.26-2.37 (m, 4H), 2.63-2.72 (m, 2H), 3.28-3.35 (m, 2H), 4.58-4.64 (m, 1H), 7.18-7.44 (m, 11H), 7.56 (d, J=7.80 Hz, 1H) ppm.

LCMS: APCI ESI m/z 592 [M+H]⁺

### Example 20

### Biphenyl-2-yl-carbamic acid 1-[9-(2-fluoro-4-hydroxy-benzoylamino)-nonyl]-piperidin-4-yl ester

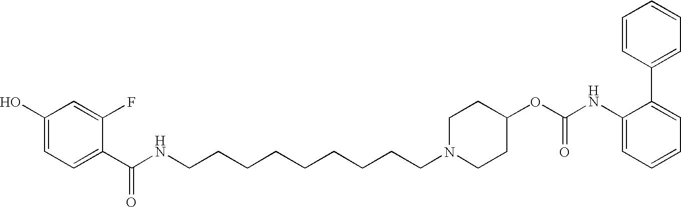

The title compound was prepared from biphenyl-2-yl-carbamic acid 1-(9-amino-nonyl)-piperidin-4-yl ester (Preparation 2, 159 mg, 0.363 mmol) and 2-fluoro-4-hydroxybenzoic acid (51.0 mg, 0.327 mmol) using the same method as described in example 19, as a colourless glass, 70% yield, 147 mg.

¹H NMR (400 MHz, METHANOL-d₄) δ=1.26-1.41 (m, 10H), 1.47-1.54 (m, 2H), 1.56-1.69 (m, 4H), 1.79-1.89 (m, 2H), 2.26-2.37 (m, 4H), 2.63-2.72 (m, 2H), 3.30-3.32 (m, 2H), 4.58-4.64 (m, 1H), 6.52-6.55 (m, 1H), 6.63-6.66 (m, 1H), 7.24-7.44 (m, 8H), 7.56 (d, J=7.80 Hz, 1H) 7.59-7.63 (m, 1H) ppm.

LCMS: APCI ESI m/z 576 [M+H]⁺

### Example 21

### Biphenyl-2-yl-carbamic acid 1-[9-(2-chloro-4-hydroxy-benzoylamino)-nonyl]-piperidin-4-yl ester

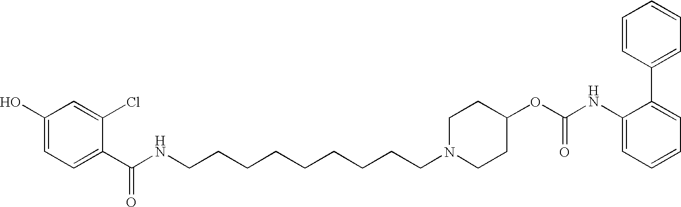

The title compound was prepared from biphenyl-2-yl-carbamic acid 1-(9-amino-nonyl)-piperidin-4-yl ester (Preparation 2, 159 mg, 0.363 mmol) and 2-chloro-4-hydroxybenzoic acid hydrate (62.3 mg, 0.327 mmol) using the same method as described in example 19, as a colourless glass, 75% yield, 162 mg.

¹H NMR (400 MHz, METHANOL-d₄) δ=1.27-1.44 (m, 10H), 1.47-1.54 (m, 2H), 1.56-1.68 (m, 4H), 1.80-1.90 (m, 2H), 2.27-2.37 (m, 4H), 2.65-2.72 (m, 2H), 3.30-3.31 (m, 2H), 4.56-4.65 (m, 1H), 6.72-6.75 (m, 1H), 6.82-6.83 (m, 1H), 7.23-7.44 (m, 9H), 7.54-7.56 (m, 1H) ppm.

### LCMS: ESI m/z 592 [M+H]⁺

### Example 22

### Biphenyl-2-yl-carbamic acid 1-[9-(3,5-dichloro-4-hydroxy-benzylamino)-nonyl]-piperidin-4-yl ester

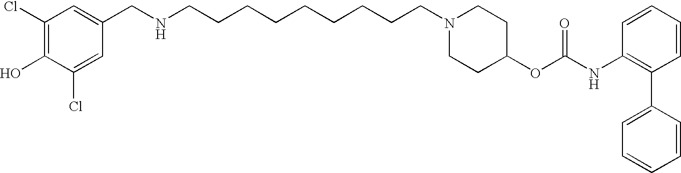

Biphenyl-2-yl-carbamic acid 1-(9-amino-nonyl)-piperidin-4-yl ester (Preparation 2, 0.5 g) and 3,5-dichloro-4-hydroxybenzaldehyde (0.187 g) were dissolved in dichloromethane (9 ml) at room temperature. Acetic acid (single drop) was added, followed by sodium triacetoxyborohydride in three portions of 100 mg each, approximately 20 minutes apart. The resulting mixture was stirred at room temperature under nitrogen for 21 hours, then partitioned between dichloromethane (20 mL) and saturated aqueous sodium bicarbonate solution (20 mL). The organic phase was separated, washed with brine (2×10 ml), dried over sodium sulphate, filtered and evaporated in vacuo to give a white solid that was purified by column chromatography on silica gel using dichloromethane:methanol:880 ammonia, 96:4:0.5 to 90:10:0.5 (by volume), as eluant to give the title compound as a white solid, 312 mg.

¹H NMR (400 Mhz, CD₃OD) δ=1.24-1.70 (m, 14H), 1.83-1.94 (m, 2H), 2.24-2.38 (m, 4H), 2.61-2.63 (m, 2H), 2.63-2.75 (m, 2H), 2.81-2.86 (m, 2H), 3.82 (s, 2H), 4.60-4.64 (m, 1H), 7.19 (s, 2H), 7.23-7.44 (m, 8H), 7.52-7.60 (d, 1H) ppm.

Alternatively, the title compound was also isolated by the following procedure:

A solution of the title compound (306 mg) in methanol (5 ml) was heated to give a clear solution and then allowed to cool to room temperature. The resulting solid was filtered and dried in vacuo to afford the title compound as a crystalline white solid, 190 mg.

¹H NMR (400 MHz, DMSO-D) δ=: 1.15-1.28 (m, 10H), 1.32-1.47 (m, 6H), 1.67-1.75 (m, 2H), 2.01-2.10 (m, 2H), 2.17-2.23 (m, 2H), 2.54-2.60 (m, 2H), 3.16 (s, 2H), 3.60 (s, 2H), 4.39-4.47 (m, 1H), 7.19 (s, 2H), 7.25-7.43 (m, 9H), 8.60 (s, 1H) ppm.

### Example 22a

### Biphenyl-2-yl-carbamic acid 1-[9-(3,5-dichloro-4-hydroxy-benzylamino)-nonyl]-piperidin-4-yl ester; naphthalene-1,5-disulfonate salt

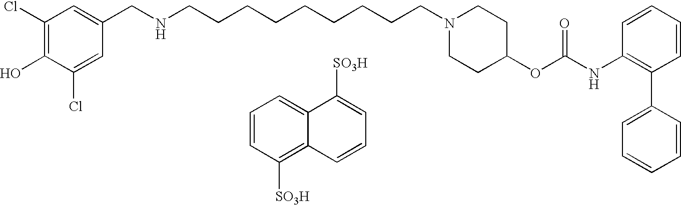

Biphenyl-2-yl-carbamic acid 1-[9-(3,5-dichloro-4-hydroxy-benzylamino)-nonyl]-piperidin-4-yl ester (Example 22, 306 mg) was heated in methanol (3 ml) to give a clear solution, followed by addition of a solution of naphthalene-1,5-disulfonic acid (180 mg) in methanol (1 ml). After 2 hours at room temperature, the resulting solid was filtered and dried in vacuo to afford the title compound as a crystalline solid, 412 mg.

¹H NMR (400 MHz, DMSO-d) δ=1.13-1.25 (m, 10H), 1.48-1.58 (m, 4H), 1.73-2.03 (m, 2H), 2.79-3.03 (m, 6H), 3.27-3.50 (m, 4H), 4.03-4.08 (t, 2H), 4.72-4.77 (m, 1H), 7.30-7.45 (m, 11H), 7.54 (m, 2H), 7.93-7.95 (d, 2H), 8.54-8.61 (bs, 2H), 8.86-8.88 (d, 2H) ppm.

### Example 23

### Biphenyl-2-yl-carbamic acid 1-[9-(2,3-difluoro-4-hydroxy-benzoylamino)-nonyl]-piperidin-4-yl ester

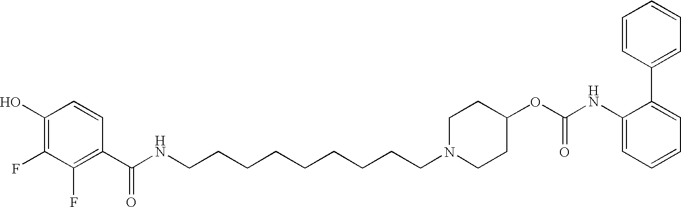

To a solution of biphenyl-2-yl-carbamic acid 1-(9-amino-nonyl)-piperidin-4-yl ester dihydrochloride salt (Preparation 2, 35 mg, 68.5 μmol) in dichloromethane (5 ml), was added triethylamine (28.7 μl, 206 □mol), 2,3-difluoro-4-hydroxybenzoic acid (12.5 mg, 72 μmol), 1-hydroxy benzotriazole monohydrate (12.6 mg, 82.3 μmol) and (3-(dimethylamino)propyl)ethyl carbodiimide hydrochloride (15.8 mg, 82.3 μmol) and stirred at room temperature for 18 hours. The resulting clear solution was diluted with dichloromethane (5 ml) and saturated aqueous sodium hydrogen carbonate solution (5 ml) and stirred vigorously for 10 minutes. The resulting biphasic solution was separated using a phase separation cartridge and the organic layer concentrated to give a white foam. The residue was purified by HPLC method D to afford the title compound.

LCMS Method D: RT 2.61 min (100% area) ES m/z 594.307 [M+H]⁺

### Example 24

### Biphenyl-2-yl-carbamic acid 1-[9-(2-hydroxy-5-chloro-benzoylamino)-nonyl]-piperidin-4-yl ester

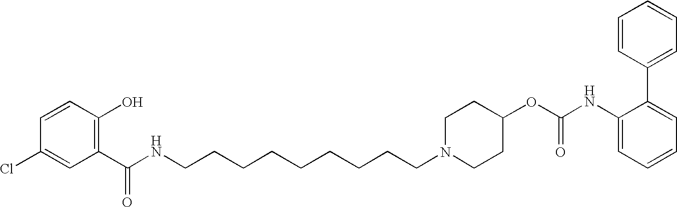

The title compound was prepared from biphenyl-2-yl-carbamic acid 1-(9-amino-nonyl)-piperidin-4-yl ester dihydrochloride salt (Preparation 2, 35 mg, 68.5 μmol) and 5-chlorosalicylic acid (12.4 mg, 72 μmol) using the same method as described in example 23 to afford the crude product as an orange gum. The residue was purified by HPLC method D to afford the title compound.

LCMS Method D: RT 2.87 min (100% area)

ES m/z 590.286 [M+H]⁺

### Example 25

### Biphenyl-2-yl-carbamic acid 1-[9-(4-hydroxy-3,5-dichloro-benzoylamino)-nonyl]-piperidin-4-yl ester

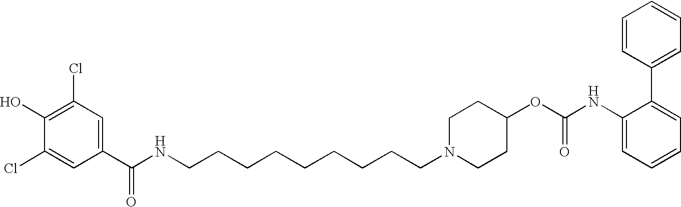

The title compound was prepared from biphenyl-2-yl-carbamic acid 1-(9-amino-nonyl)-piperidin-4-yl ester dihydrochloride salt (Preparation 2, 35 mg, 68.5 μmol) and 4-hydroxy-3,5-dichloro-benzoic acid (14.9 mg, 72 μmol) using the same method as described in example 23 to afford the crude product as a white foam. The residue was purified by HPLC method D to afford the title compound.

LCMS Method D: RT 2.68 min (100% area)

ES m/z 626.247 [M+H]⁺

### Example 26

### Biphenyl-2-yl-carbamic acid 1-[9-(2-hydroxy-4-fluoro-benzoylamino)-nonyl]-piperidin-4-yl ester

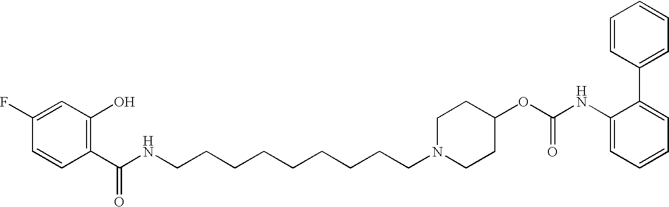

The title compound was prepared from biphenyl-2-yl-carbamic acid 1-(9-amino-nonyl)-piperidin-4-yl ester dihydrochloride salt (Preparation 2, 35 mg, 68.5 μmol) and 2-hydroxy-4-fluoro-benzoic acid (10.7 mg, 72 μmol) using the same method as described in example 23 to afford the crude product as a colourless gum. The residue was purified by HPLC method D to afford the title compound.

LCMS Method D: RT 2.82 min (100% area)

ES m/z 576.316 [M+H]⁺

### Example 27

### Biphenyl-2-yl-carbamic acid 1-[9-(3,5-difluoro-4-hydroxy-benzylamino)-nonyl]-piperidin-4-yl ester

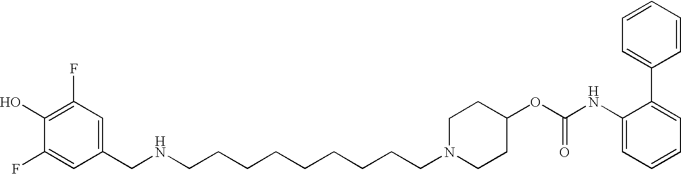

To a solution of biphenyl-2-yl-carbamic acid 1-(9-amino-nonyl)-piperidin-4-yl ester dihydrochloride salt (Preparation 2, 35 mg, 68.5 μmol) and triethylamine (9.5 μl, 68.5 μmol) in ethanol (1 ml) were added 3,5-difluoro-4-hydroxybenzaldehyde (10.8 mg, 68.5 μmol), acetic acid (in excess of 4 μl, 68.5 μmol) and sodium sulfate, and stirred under nitrogen at room temperature for 1 hour. Sodium cyanoborohydride was then added (8.6 mg, 137 μmol) and the mixture stirred under nitrogen at room temperature for 18 hours. The solvent was removed in vacuo and the residue was dissolved with dichloromethane (3 ml) and sodium hydrogen carbonate solution (3 ml). The resulting biphasic solution was stirred vigorously for 10 minutes then separated using a phase separation cartridge and the organic layer concentrated in vacuo to yield a yellow gum. The residue was purified by HPLC method A to afford the title compound.

LCMS Method A: RT 2.42 min (100% area)

ES m/z 580.327[M+H]⁺

### Example 28

### Biphenyl-2-yl-carbamic acid 1-[9-(2-hydroxy-4,5-dichloro-benzylamino)-nonyl]-piperidin-4-yl ester

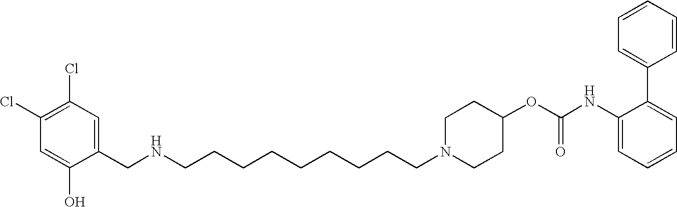

The title compound was prepared from biphenyl-2-yl-carbamic acid 1-(9-amino-nonyl)-piperidin-4-yl ester dihydrochloride salt (Preparation 2, 35 mg, 68.5 μmol) and 2-hydroxy-4,5-dichloro-benzaldehyde (13.1 mg, 68.5 μmol) using the same method as described in example 27 to afford the crude product as a yellow gum. The residue was purified by HPLC method A to afford the title compound.

LCMS Method A: RT 2.34 min (100% area)

ES m/z 612.268 [M+H]⁺

### Example 29

### Biphenyl-2-yl-carbamic acid 1-[9-(2-hydroxy-3,5-difluoro-benzylamino)-nonyl]-piperidin-4-yl ester

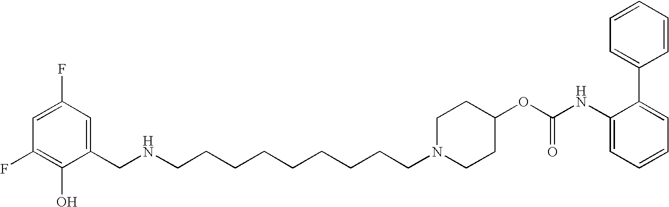

The title compound was prepared from biphenyl-2-yl-carbamic acid 1-(9-amino-nonyl)-piperidin-4-yl ester dihydrochloride salt (Preparation 2, 35 mg, 68.5 μmol) and 3,5-difluoro-salicylaldehyde (10.8 mg, 68.5 μmol) using the same method as described in example 27 to afford the crude product as a yellow gum. The residue was purified by HPLC method A to afford the title compound.

LCMS Method A: RT 2.28 min (100% area)

ES m/z 580.327 [M+H]⁺

### Example 30

### Biphenyl-2-yl-carbamic acid 1-[9-(2-hydroxy-3-fluoro-benzylamino)-nonyl]-piperidin-4-yl ester

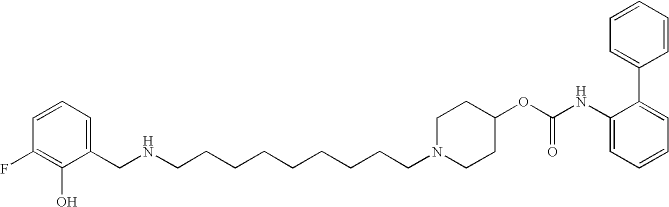

The title compound was prepared from biphenyl-2-yl-carbamic acid 1-(9-amino-nonyl)-piperidin-4-yl ester dihydrochloride salt (Preparation 2, 35 mg, 68.5 μmol) and 2-hydroxy-3-fluoro-benzaldehyde (9.6 mg, 68.5 μmol) using the same method as described in example 27 to afford the crude product as a yellow gum. The residue was purified by HPLC method A to afford the title compound.

LCMS Method A: RT 2.19 min (100% area)

ES m/z 562.337 [M+H]+

### Example 31

### Biphenyl-2-yl-carbamic acid 1-[9-(2-hydroxy-3,5-dichloro-benzylamino)-nonyl]-piperidin-4-yl ester

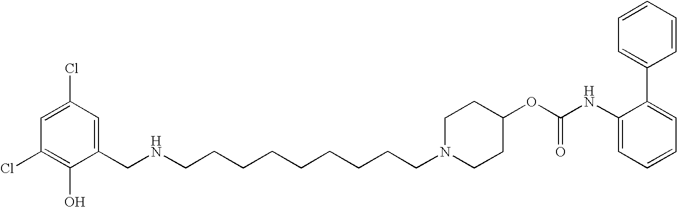

The title compound was prepared from biphenyl-2-yl-carbamic acid 1-(9-amino-nonyl)-piperidin-4-yl ester dihydrochloride salt (Preparation 2, 35 mg, 68.5 μmol) and 3,5-dichloro-salicylaldehyde (13.1 mg, 68.5 μmol) using the same method as described in example 27 to afford the crude product as a yellow gum. The residue was purified by HPLC method A to afford the title compound.

LCMS Method A: RT 2.28 min (100% area)

ES m/z 612.268 [M+H]⁺

### Example 32

### Biphenyl-2-yl-carbamic acid 1-[9-(5-chloro-2-hydroxy-benzylamino)-nonyl]-piperidin-4-yl ester

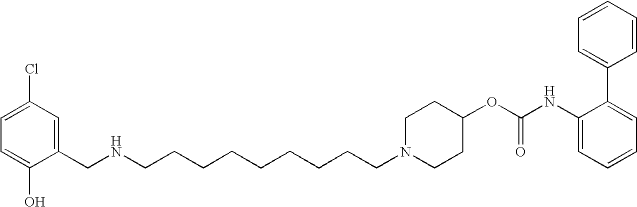

The title compound was prepared from biphenyl-2-yl-carbamic acid 1-(9-amino-nonyl)-piperidin-4-yl ester dihydrochloride salt (Preparation 2, 35 mg, 68.5 μmol) and 2-hydroxy-5-chloro-benzaldehyde (10.7 mg, 68.5 μmol) using the same method as described in example 27 to afford the crude product as a yellow gum. The residue was purified by HPLC method A to afford the title compound.

LCMS Method A: RT 2.51 min (100% area)

ES m/z 578.307 [M+H]⁺

### Example 33

### Biphenyl-2-yl-carbamic acid 1-[9-(2-hydroxy-4-chloro-5-fluoro-benzylamino)-nonyl]-piperidin-4-yl ester

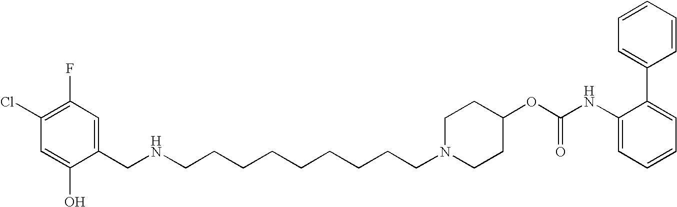

The title compound was prepared from biphenyl-2-yl-carbamic acid 1-(9-amino-nonyl)-piperidin-4-yl ester dihydrochloride salt (Preparation 2, 35 mg, 68.5 μmol) and 2-hydroxy-4-chloro-5-fluoro-benzaldehyde (Preparation 8, 12.0 mg, 68.5 μmol) using the same method as described in example 27 to afford the crude product as a yellow gum. The residue was purified by HPLC method A to afford the title compound.

LCMS Method A: RT 2.34 min (100% area) ES m/z 596.298 [M+H]⁺

### Example 34

### Biphenyl-2-yl-carbamic acid 1-[9-(3-chloro-4-hydroxy-benzylamino)-nonyl]-piperidin-4-yl ester

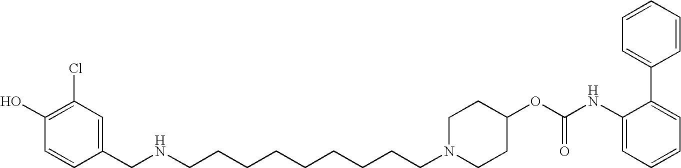

The title compound was prepared from biphenyl-2-yl-carbamic acid 1-(9-amino-nonyl)-piperidin-4-yl ester dihydrochloride salt (Preparation 2, 35 mg, 68.5 μmol) and 3-chloro-4-hydroxy-benzaldehyde (10.7 mg, 68.5 μmol) using the same method as described in example 27 to afford the crude product as a yellow gum. The residue was purified by HPLC method A to afford the title compound.

LCMS Method A: RT 2.20 min (100% area)

ES m/z 578.307 [M+H]⁺

### Example 35

### Biphenyl-2-yl-carbamic acid 1-[9-(2-hydroxy-5-fluoro-benzylamino)-nonyl]-piperidin-4-yl ester

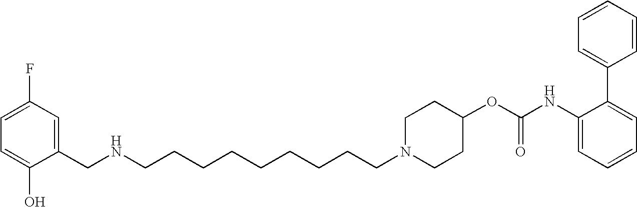

The title compound was prepared from biphenyl-2-yl-carbamic acid 1-(9-amino-nonyl)-piperidin-4-yl ester dihydrochloride salt (Preparation 2, 35 mg, 68.5 μmol) and 2-hydroxy-5-fluoro-benzaldehyde (9.6 mg, 68.5 μmol) using the same method as described in example 27 to afford the crude product as a yellow gum. The residue was purified by HPLC method A to afford the title compound.

LCMS Method A: RT 2.27 min (100% area)

ES m/z 562.337 [M+H]⁺

### Example 36

### Biphenyl-2-yl-carbamic acid 1-[9-(3-hydroxy-4-fluoro-benzylamino)-nonyl]-piperidin-4-yl ester

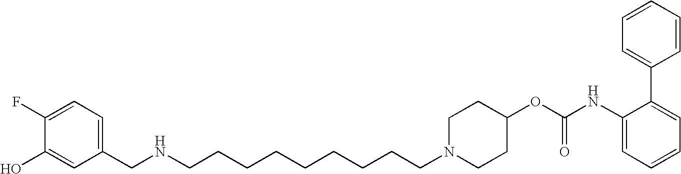

The title compound was prepared from biphenyl-2-yl-carbamic acid 1-(9-amino-nonyl)-piperidin-4-yl ester dihydrochloride salt (Preparation 2, 100 mg, 196 μmol) and 3-hydroxy-4-fluoro-benzaldehyde (27.4 mg, 196 μmol) using the same method as described in example 27 to afford the crude product as a yellow gum (110 mg). The residue was purified using silica gel column chromatography eluting with ethyl acetate:methanol:0.88 ammonia (95:5:0.5 to 90:10:1.0, by volume) to afford the title compound as a colourless gum, 68% yield, 75 mg.

¹H NMR (400 MHz, METHANOL-d₄) δ=1.27-1.34 (m, 10H), 1.46-1.55 (m, 4H), 1.58-1.65 (m, 2H), 1.81-1.88 (m, 2H), 2.23-2.34 (m, 4H), 2.58-2.62 (t, 2H), 2.64-2.69 (m, 2H), 3.68 (s, 2H), 4.56-4.62 (m, 1H), 6.67-6.71 (m, 1H), 6.85-6.88 (dd, 1H), 6.93-6.98 (m, 1H), 7.23-7.44 (m, 8H), 7.53-7.57 (m, 1H) ppm.

LCMS: APCI ESI m/z 562 [M+H]⁺

### Example 37

### Biphenyl-2-yl-carbamic acid 1-[9-(2,4-dichloro-3-hydroxy-benzylamino)-nonyl]-piperidin-4-yl ester

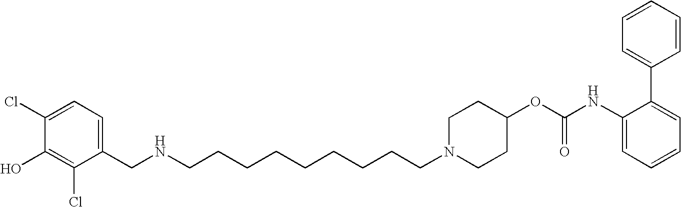

The title compound was prepared from biphenyl-2-yl-carbamic acid 1-(9-amino-nonyl)-piperidin-4-yl ester dihydrochloride salt (Preparation 2, 100 mg, 196 μmol) and 3-hydroxy-2,4-dichlorobenzaldehyde (37.4 mg, 196 μmol) using the same method as described in example 27 to afford the crude product as an off-white foam (127 mg). The residue was purified using silica gel column chromatography eluting with ethyl acetate:methanol:0.88 ammonia (95:5:0.5 to 85:15:1.5, by volume) to afford the title compound as an orange gum, 76% yield, 91 mg.

¹H NMR (400 MHz, METHANOL-d₄) δ ppm 1.26-1.40 (m, 10H), 1.47-1.54 (m, 2H), 1.58-1.66 (m, 4H), 1.83-1.91 (m, 2H), 2.38-2.42 (m, 4H), 2.74-2.78 (m, 2H), 2.82-2.84 (m, 2H), 4.02 (s, 2H), 4.60-4.64 (m, 1H), 6.58-6.51 (m, 1H), 7.17-7.19 (d, 1H), 7.22-7.48 (m, 8H), 7.56-7.60 (m, 1H).

LCMS: APCI ESI m/z 612 [M+H]⁺, 610 [M⁻]

Alternatively, the title compound was prepared by the following procedure:

Biphenyl-2-yl-carbamic acid 1-(9-amino-nonyl)-piperidin-4-yl ester (Preparation 2a, 3.11 g, 7.116 mmol) was dissolved in ethanol (60 ml), to which was added 3-hydroxy-2,4-dichlorobenzaldehyde (2.04 g, 10.7 mmol) followed by titanium tetraisopropoxide (4.17 ml, 14.2 mmol). The reaction mixture was stirred at room temperature for 18 hours, then cooled to 0° C. and sodium borohydride (808 mg, 21.3 mmol) added portionwise over 30 minutes. The reaction was allowed to warm to room temperature and stirred for 4 hours. The reaction was quenched by the dropwise addition of water (10 ml) and left to stand for 18 hours at room temperature. The mixture was partitioned between dichloromethane (200 ml) and 1N aqueous hydrochloric acid. The organic layer was washed with saturated aqueous sodium bicarbonate (150 ml), brine (150 ml), dried over magnesium sulphate and concentrated in vacuo. The residue was purified using silica gel column chromatography eluting with dichloromethane:methanol:0.88 ammonia (95:5:0.5 to 90:10:1, by volume) to afford the title compound as a white foam, in 46% yield, 1.99 g.

70 mg of the title compound was dissolved in hot methanol (5 ml), then allowed to cool slowly to room temperature and left standing for 18 hours. The resulting solid was collected by filtration to yield the title compound as a white crystalline solid, 50 mg.

LCMS: APCI ESI m/z 612 [M+H]⁺, 610 [M⁻]

¹H NMR (400 MHz, METHANOL-d₄) δ=1.26-1.40 (m, 10H), 1.47-1.54 (m, 2H), 1.58-1.66 (m, 4H), 1.83-1.91 (m, 2H), 2.38-2.42 (m, 4H), 2.74-2.78 (m, 2H), 2.82-2.84 (m, 2H), 4.02 (s, 2H), 4.60-4.64 (m, 1H), 6.58-6.51 (m, 1H), 7.17-7.19 (d, 1H), 7.22-7.48 (m, 8H), 7.56-7.60 (m, 1H) ppm.

### Example 38

### Biphenyl-2-yl-carbamic acid 1-[9-(4-hydroxy-3-fluoro-benzylamino)-nonyl]-piperidin-4-yl ester

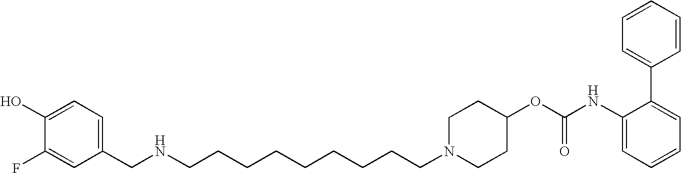

The title compound was prepared from biphenyl-2-yl-carbamic acid 1-(9-amino-nonyl)-piperidin-4-yl ester dihydrochloride salt (Preparation 2, 100 mg, 196 μmol) and 4-hydroxy-3-fluoro-benzaldehyde (27.4 mg, 196 μmol) using the same method as described in example 37, to afford the title compound as a colourless gum, 68% yield, 75 mg.

¹H NMR (400 MHz, METHANOL-d₄) δ=1.21-1.36 (m, 10H), 1.44-1.55 (m, 4H), 1.56-1.65 (m, 2H), 1.78-1.87 (m, 2H), 2.21-2.35 (m, 4H), 2.57-2.68 (m, 4H), 3.67 (s, 2H), 4.56-4.63 (m, 1H), 6.79-6.84 (m, 1H), 6.89-6.93 (m, 1H), 7.00-7.03 (m, 1H), 7.23-7.42 (m, 8H), 7.53-7.58 (m, 1H) ppm.

LCMS: APCI ESI m/z 562 [M+H]⁺, 584 [M+Na]⁺

### Example 39

### Biphenyl-2-yl-carbamic acid 1-{9-[bis-(2-chloro-3-hydroxy-benzyl)-amino]-nonyl}-piperidin-4-yl ester

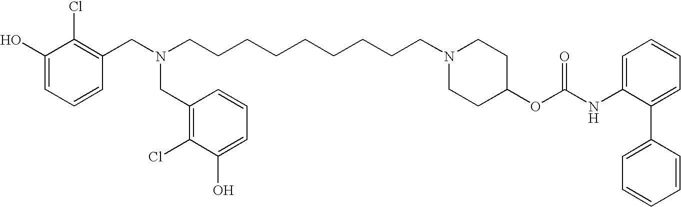

The title compound was prepared from biphenyl-2-yl-carbamic acid 1-(9-amino-nonyl)-piperidin-4-yl ester (Preparation 2, 130 mg, 297 μmol) and 2-chloro-3-hydroxybenzaldehyde (46.5 mg, 297 μmol) using the same method as described in example 2 to afford the title compound as a white foam, 15% yield, 32 mg.

¹H NMR (400 MHz, METHANOL-d₄) δ=1.16-1.32 (m, 10H), 1.45-1.55 (m, 4H), 1.58-1.69 (m, 2H), 1.79-1.92 (m, 2H), 2.24-2.37 (m, 4H), 2.45-2.48 (t, 2H), 2.63-2.74 (m, 2H), 3.67 (s, 4H), 4.57-4.64 (m, 1H), 6.77-6.79 (m, 2H), 7.02-7.07 (m, 4H), 7.23-7.44 (m, 8H), 7.53-7.57 (m, 1H) ppm.

LCMS: m/z 718-20 [M+H]+, 716-717 M−

### Example 40

### Biphenyl-2-yl-carbamic acid 1-{9-[(3-fluoro-2-hydroxy-benzyl)-methyl-amino]-nonyl}-piperidin-4-yl ester

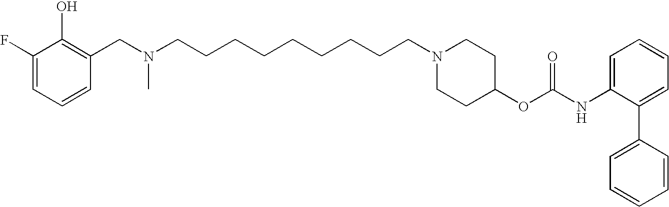

Biphenyl-2-yl-carbamic acid 1-(9-methylamino-nonyl)-piperidin-4-yl ester; dihydrochloride salt (Preparation 7, 40.0 mg, 89 μmol) was dissolved in ethanol (0.5 ml) and added to a reaction vessel containing 3-fluoro-2-hydroxybenzaldehyde (12.5 mg, 89 μmol). To the reaction mixture was then added acetic acid (5.1 μl, 90 μmol) and sodium sulphate (drying agent) and the resulting mixture allowed to stir for 30 minutes at room temperature. Sodium tri(acetoxy)borohydride (38 mg, 178 μmol) in ethanol (0.5 ml) was then added, and the reaction allowed to stir at room temperature for 18 h. Further sodium tri(acetoxy)borohydride (19 mg, 89 μmol) was added and the reaction stirred for a further 24 hours. The solvents were removed in vacuo and the residue partitioned between dichloromethane (2 ml) and saturated aqueous sodium hydrogen carbonate solution (2 ml). The aqueous phase was separated and extracted with further dichloromethane (1 ml). The combined organic layers were concentrated in vacuo and the residue purified by HPLC method A to afford the title compound.

LCMS Method A: RT 2.28 min (100% area)

ES m/z 576 [M+H]⁺.

### Example 41

### Biphenyl-2-yl-carbamic acid 1-{9-[(4,5-dichloro-2-hydroxy-benzyl)-methyl-amino]-nonyl}-piperidin-4-yl ester

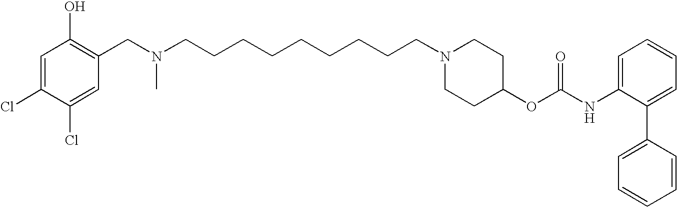

The title compound was prepared from biphenyl-2-yl-carbamic acid 1-(9-methylamino-nonyl)-piperidin-4-yl ester; dihydrochloride salt (Preparation 7, 40.0 mg, 89 μmol) and 4,5-dichloro-2-hydroxy benzaldehyde (17.0 mg, 89 μmol) using the same method as described in example 40.

LCMS Method A: RT 2.27 min (100% area)

ES m/z 626 [M+H]⁺.

### Example 42

### Biphenyl-2-yl-carbamic acid 1-{9-[(4-fluoro-3-hydroxy-benzyl)-methyl-amino]-nonyl}-piperidin-4-yl ester

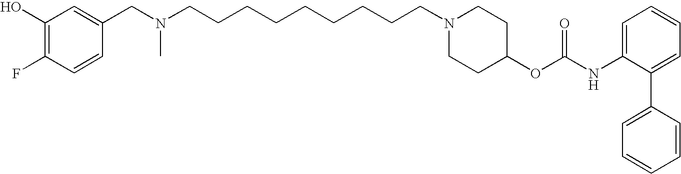

Biphenyl-2-yl-carbamic acid 1-(9-methylamino-nonyl)-piperidin-4-yl ester; dihydrochloride salt (Preparation 7, 150 mg, 0.332 mmol) was dissolved in dichloromethane (3 ml). To this was added 4-fluoro-3-hydroxybenzaldehyde (Bioorg. Med. Chem., 2001, 9, 677; 51.1 mg, 0.365 mmol), acetic acid (19.0 μl, 0.332 mmol) and sodium tri(acetoxy)borohydride (141 mg, 0.664 mmol). The resulting mixture was stirred at room temperature for 18 hours. The reaction was quenched with 2M aqueous sodium carbonate solution (5 ml) and then partitioned between ethyl acetate (20 ml) and water (20 ml). The organic layer was washed with brine (20 ml), dried (sodium sulphate) and concentrated in vacuo. The crude residue was purified by column chromatography on silica gel eluting with dichloromethane:methanol:880 ammonia (100:0:0 to 90:10:1, by volume), to furnish the title compound as a colourless oil, in 28% yield, 53 mg.

LCMS: ESI m/z 576 [M+H]⁺

¹H NMR (400 MHz, METHANOL-d₄) δ=1.27-1.34 (m, 10H), 1.46-1.55 (m, 4H), 1.59-1.67 (m, 2H), 1.81-1.89 (m, 2H), 2.20 (s, 3H), 2.25-2.39 (m, 6H), 2.63-2.71 (m, 2H), 3.43 (s, 2H), 4.57-4.63 (m, 1H), 6.69-6.74 (m, 1H), 6.88 (d, 1H), 6.94-6.99 (m, 1H), 7.23-7.44 (m, 8H), 7.55 (d, 1H) ppm.

### Example 43

### Biphenyl-2-yl-carbamic acid 1-{9-[(4-chloro-5-fluoro-2-hydroxy-benzyl)-methyl-amino]-nonyl}-piperidin-4-yl ester

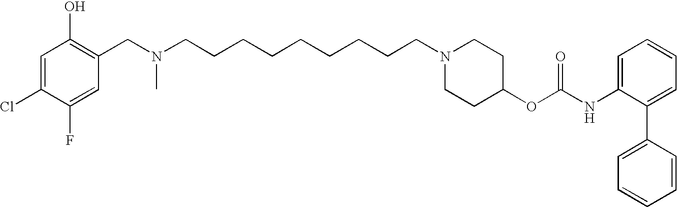

Biphenyl-2-yl-carbamic acid 1-(9-methylamino-nonyl)-piperidin-4-yl ester; dihydrochloride salt (Preparation 7, 200 mg, 0.443 mmol) was dissolved in dichloroethane (5 ml). To this was added 4-chloro-5-fluoro-2-hydroxybenzaldehyde (Preparation 8, 73.7 mg, 0.422 mmol) and the reaction mixture stirred at room temperature for 1 hour. Sodium tri(acetoxy)borohydride (125 mg, 0.591 mmol) was added and the mixture stirred at room temperature for a further 18 hours. The reaction was quenched by dropwise addition of water (1 ml) and the solvent was removed in vacuo. The residue was partitioned between dichloromethane (20 ml) and saturated aqueous sodium hydrogen carbonate solution (20 ml), the organic layer was dried (magnesium sulphate) and then concentrated in vacuo. The crude residue was purified by column chromatography on silica gel eluting with dichloromethane:methanol:880 ammonia (100:0:0 to 95:5:0.5, by volume), to furnish the title compound as a colourless oil, in 45% yield, 121 mg.

LCMS: ESI m/z 610 [M+H]⁺

¹H NMR (400 MHz, METHANOL-d₄) δ=1.28-1.35 (m, 10H), 1.48-1.68 (m, 6H), 1.82-1.89 (m, 2H), 2.29 (s, 3H), 2.32-2.40 (m, 4H), 2.48-2.52 (m, 2H), 2.67-2.74 (m, 2H), 3.68 (s, 2H), 4.59-4.65 (m, 1H), 6.77 (d, 1H), 6.93 (d, 1H), 7.23-7.44 (m, 8H), 7.56 (d, 1H) ppm.

### Example 44

### Biphenyl-2-yl-carbamic acid 1-{9-[(3-chloro-4-hydroxy-benzyl)-methyl-amino]-nonyl}-piperidin-4-yl ester

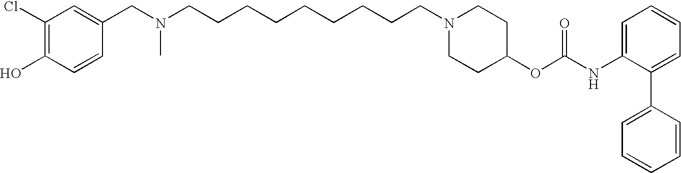

The title compound was prepared from biphenyl-2-yl-carbamic acid 1-(9-methylamino-nonyl)-piperidin-4-yl ester; dihydrochloride salt (Preparation 7, 150 mg, 0.332 mmol) and 3-chloro-4-hydroxybenzaldehyde (57.2 mg, 0.365 mmol) using the same method as described in example 42 to afford the title compound as a white foam, in 72% yield, 142 mg.

LCMS: ESI m/z 592 [M+H]⁺

¹H NMR (400 MHz, METHANOL-d₄) δ=1.26-1.33 (m, 10H), 1.46-1.55 (m, 4H), 1.57-1.67 (m, 2H), 1.80-1.89 (m, 2H), 2.19 (s, 3H), 2.24-2.39 (m, 6H), 2.62-2.71 (m, 2H), 3.42 (s, 2H), 4.56-4.63 (m, 1H), 6.85 (d, 1H), 7.05 (d, 1H), 7.23-7.44 (m, 9H), 7.55 (d, 1H) ppm.

### Example 45

### Biphenyl-2-yl-carbamic acid 1-{9-[(3-fluoro-4-hydroxy-benzyl)-methyl-amino]-nonyl}-piperidin-4-yl ester

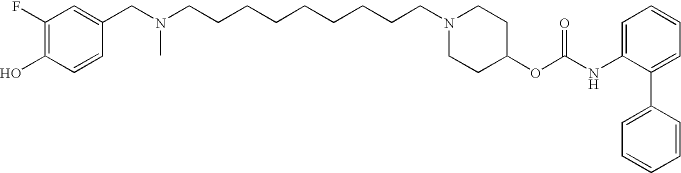

The title compound was prepared from biphenyl-2-yl-carbamic acid 1-(9-methylamino-nonyl)-piperidin-4-yl ester; dihydrochloride salt (Preparation 7, 150 mg, 0.332 mmol) and 3-fluoro-4-hydroxybenzaldehyde (51.1 mg, 0.365 mmol) using the same method as described in example 42 to afford the title compound as a white foam, in 19% yield, 37 mg.

LCMS: ESI m/z 576 [M+H]⁺

¹H NMR (400 MHz, METHANOL-d₄) δ=1.26-1.34 (m, 10H), 1.47-1.56 (m, 4H), 1.58-1.67 (m, 2H), 1.81-1.89 (m, 2H), 2.20 (s, 3H), 2.25-2.40 (m, 6H), 2.63-2.72 (m, 2H), 3.44 (s, 2H), 4.57-4.65 (m, 1H), 6.82-6.92 (m, 2H), 7.02 (d, 1H), 7.23-7.44 (m, 8H), 7.55 (d, 1H) ppm.

### Example 46

### Biphenyl-2-yl-carbamic acid 1-{9-[(5-chloro-2-hydroxy-benzyl)-methyl-amino]-nonyl}-piperidin-4-yl ester

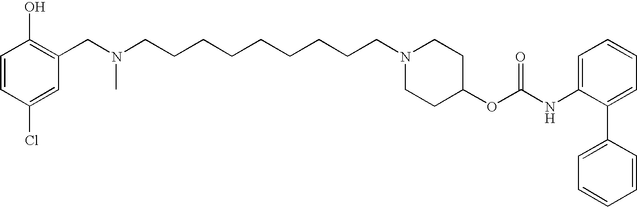

The title compound was prepared from biphenyl-2-yl-carbamic acid 1-(9-methylamino-nonyl)-piperidin-4-yl ester; dihydrochloride salt (Preparation 7, 40.0 mg, 89 μmol) and 5-chloro-2-hydroxy benzaldehyde (13.9 mg, 89 μmol) using the same method as described in example 40.

LCMS Method A: RT 2.43 min (100% area)

ES m/z 592 [M+H]⁺.

### Example 47

### Biphenyl-2-yl-carbamic acid 1-{9-[(5-fluoro-2-hydroxy-benzyl)-methyl-amino]-nonyl}-piperidin-4-yl ester

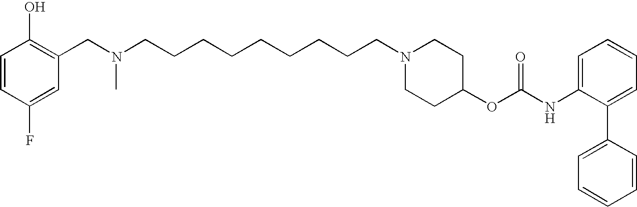

The title compound was prepared from biphenyl-2-yl-carbamic acid 1-(9-methylamino-nonyl)-piperidin-4-yl ester; dihydrochloride salt (Preparation 7, 40.0 mg, 89 μmol) and 5-fluoro-2-hydroxy benzaldehyde (12.5 mg, 89 μmol) using the same method as described in example 40.

LCMS Method A: RT 2.27 min (100% area)

ES m/z 576 [M+H]⁺.

### Example 48

### Biphenyl-2-yl-carbamic acid 1-{9-[(2-chloro-3-hydroxy-benzyl)-methyl-amino]-nonyl}-piperidin-4-yl ester

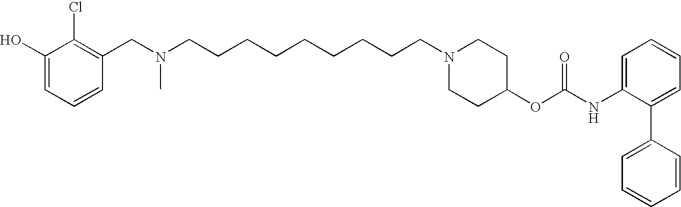

Biphenyl-2-yl-carbamic acid 1-(9-methylamino-nonyl)-piperidin-4-yl ester (Preparation 7a, 1.116 g, 2.583 mmol) was dissolved in dichloroethane (25 ml) and to this was added 2-chloro-3-hydroxybenzaldehyde. The resulting mixture was stirred at room temperature for 15 minutes prior to the addition of sodium tri(acetoxy)borohydride (733 mg, 3.46 mmol). The reaction was then stirred at room temperature for 18 hours. The reaction was quenched by the addition of water (2 ml) and the solvents were removed in vacuo. The residue was partitioned between dichloromethane:methanol (50 ml, 95:5 by volume) and water (20 ml). The organic layer was dried (magnesium sulphate) and concentrated in vacuo. The residue was purified using silica gel column chromatography eluting with dichloromethane:methanol:0.88 ammonia (97:3:0.3 to 94:6:0.6, by volume) to afford the title compound as a colourless glass, in 77% yield, 1.12 g.

LCMS: ESI m/z 592 [M+H]⁺

¹H NMR (400 MHz, METHANOL-d₄) δ=1.26-1.35 (m, 10H), 1.45-1.66 (m, 6H), 1.81-1.87 (m, 2H), 2.23 (s, 3H), 2.24-2.34 (m, 4H), 2.42-2.45 (m, 2H), 2.62-2.70 (m, 2H), 3.61 (s, 2H), 4.57-4.63 (m, 1H), 6.83 (d, 1H), 6.92 (d, 1H), 7.06-7.10 (m, 1H), 7.22-7.44 (m, 8H), 7.55 (d, 1H) ppm.

### Example 48a

### Biphenyl-2-yl-carbamic acid 1-{9-[(2-chloro-3-hydroxy-benzyl)-methyl-amino]-nonyl}-piperidin-4-yl ester; naphthalene-1,5-disulfonate salt

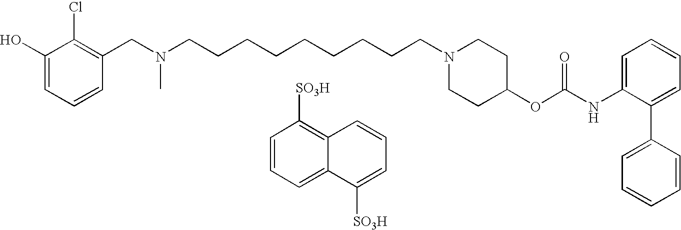

Biphenyl-2-yl-carbamic acid 1-{9-[(2-chloro-3-hydroxy-benzyl)-methyl-amino]-nonyl}-piperidin-4-yl ester (Example 48, 87 mg, 0.15 mmol) was dissolved in methanol (5 ml), to which was added naphthalene-1,5-disulfonic acid (42.4 mg, 0.15 mmol). The mixture was allowed to stir for 2.5 hours and the solvent reduced in vacuo resulting in a white precipitate that was collected by filtration to give the title compound as a white solid, in 78% yield, 101 mg.

### Example 49

### Biphenyl-2-yl-carbamic acid 1-{9-[(3-chloro-5-fluoro-2-hydroxy-benzyl)-methyl-amino]-nonyl}-piperidin-4-yl ester

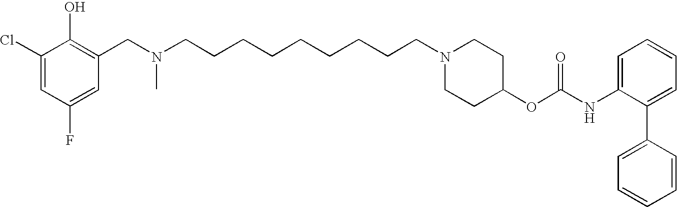

The title compound was prepared from biphenyl-2-yl-carbamic acid 1-(9-methylamino-nonyl)-piperidin-4-yl ester; dihydrochloride salt (Preparation 7, 40.0 mg, 89 μmol) and 3-chloro-5-fluoro-2-hydroxybenzaldehyde (15.5 mg, 89 μmol) using the same method as described in example 40.

LCMS Method A: RT 2.37 min (100% area)

ES m/z 610 [M+H]⁺.

### Example 50

### Biphenyl-2-yl-carbamic acid 1-{9-[(3,5-dichloro-4-hydroxy-benzyl)-methyl-amino]-nonyl}-piperidin-4-yl ester

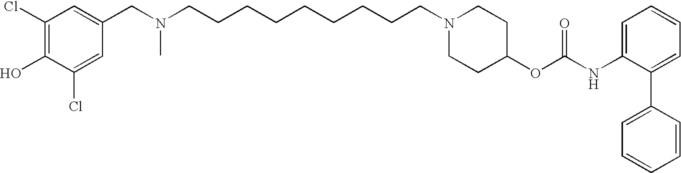

The title compound was prepared from Biphenyl-2-yl-carbamic acid 1-(9-methylamino-nonyl)-piperidin-4-yl ester; dihydrochloride salt (Preparation 7, 150 mg, 0.332 mmol) and 3,5-dichloro-4-hydroxybenzaldehyde (69.7 mg, 0.365 mmol) using the same method as described in example 42 to afford the title compound as a white foam, in 44% yield, 91 mg.

¹H NMR (400 MHz, METHANOL-d₄) δ=1.23-1.34 (m, 10H), 1.45-1.56 (m, 4H), 1.64-1.75 (m, 2H), 1.89-1.97 (m, 2H), 2.34 (s, 3H), 2.42-2.52 (m, 6H), 2.72-2.81 (m, 2H), 3.53 (s, 2H), 4.62-4.69 (m, 1H), 7.19 (s, 2H), 7.23-7.44 (m, 8H), 7.55 (d, 1H) ppm.

LCMS: ESI m/z 624 [M−H]⁻

### Example 51

### Biphenyl-2-yl-carbamic acid 1-{9-[(2-fluoro-3-hydroxy-benzyl)-methyl-amino]-nonyl}-piperidin-4-yl ester

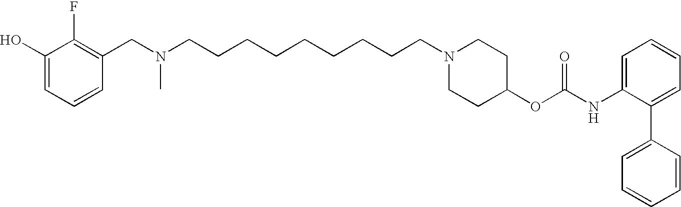

The title compound was prepared from Biphenyl-2-yl-carbamic acid 1-(9-methylamino-nonyl)-piperidin-4-yl ester; dihydrochloride salt (Preparation 7, 200 mg, 0.443 mmol) and 2-fluoro-3-hydroxybenzaldehyde (Journal of Medicinal Chemistry, 1986, 29(10), 1982-8; 59.1 mg, 0.422 mmol) using the same method as described in example 43. The crude product was purified using silica gel column chromatography eluting with ethyl acetate:methanol:0.88 ammonia (100:0:0 to 95:5:0.5, by volume) to afford the title compound as a colourless oil, in 61% yield, 156 mg.

LCMS: ESI m/z 576 [M+H]⁺

¹H NMR (400 MHz, METHANOL-d₄) δ=1.27-1.33 (m, 10H), 1.46-1.66 (m, 6H), 1.81-1.87 (m, 2H), 2.21 (s, 3H), 2.24-2.34 (m, 4H), 2.37-2.41 (m, 2H), 2.61-2.69 (m, 2H), 3.56 (s, 2H), 4.56-4.63 (m, 1H), 6.76-6.79 (m, 1H), 6.81-6.86 (m, 1H), 6.90-6.94 (m, 1H), 7.22-7.44 (m, 8H), 7.56 (d, 1H) ppm.

### Example 52

### Biphenyl-2-yl-carbamic acid 1-{9-[(3,5-dichloro-2-hydroxy-benzyl)-methyl-amino]-nonyl}-piperidin-4-yl ester

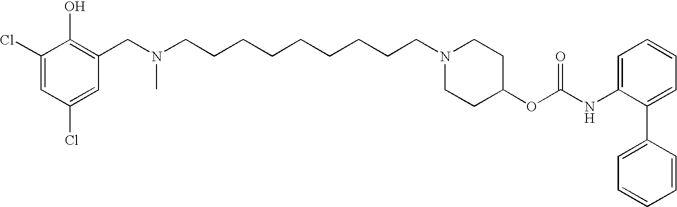

The title compound was prepared from biphenyl-2-yl-carbamic acid 1-(9-methylamino-nonyl)-piperidin-4-yl ester; dihydrochloride salt (Preparation 7, 40.0 mg, 89 μmol) and 3,5-dichloro-2-hydroxybenzaldehyde (17.0 mg, 89 μmol) using the same method as described in example 40.

LCMS Method A: RT 2.34 min (100% area)

ES m/z 626 [M+H]⁺.

### Example 53

### Biphenyl-2-yl-carbamic acid 1-{9-[(3,5-difluoro-4-hydroxy-benzyl)-methyl-amino]-nonyl}-piperidin-4-yl ester

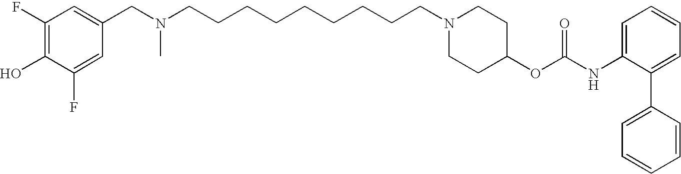

The title compound was prepared from Biphenyl-2-yl-carbamic acid 1-(9-methylamino-nonyl)-piperidin-4-yl ester; dihydrochloride salt (Preparation 7, 150 mg, 0.332 mmol) and 3,5-difluoro-4-hydroxybenzaldehyde (57.7 mg, 0.365 mmol) using the same method as described in example 42 to afford the title compound as a white foam, 41% yield, 80 mg.

LCMS: ESI m/z 592 [M−H]

¹H NMR (400 MHz, METHANOL-d₄) δ=1.25-1.34 (m, 10H), 1.46-1.56 (m, 4H), 1.61-1.71 (m, 2H), 1.83-1.92 (m, 2H), 2.26 (s, 3H), 2.35-2.46 (m, 6H), 2.68-2.77 (m, 2H), 3.48 (s, 2H), 4.60-4.66 (m, 1H), 6.85 (d, 2H), 7.22-7.44 (m, 8H), 7.55 (d, 1H) ppm.

### Example 54

### Biphenyl-2-yl-carbamic acid 1-{9-[(2,4-dichloro-3-hydroxy-benzyl)-methyl-amino]-nonyl}-piperidin-4-yl ester

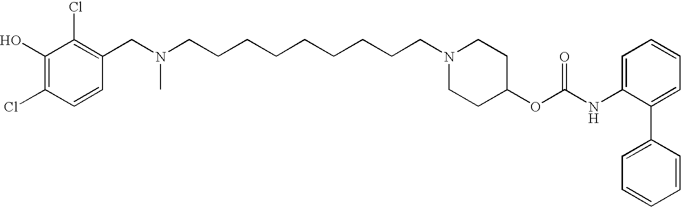

The title compound was prepared from Biphenyl-2-yl-carbamic acid 1-(9-methylamino-nonyl)-piperidin-4-yl ester; dihydrochloride salt (Preparation 7, 200 mg, 0.443 mmol) and 2,4-dichloro-3-hydroxybenzaldehyde (Preparation 10, 80.6 mg, 0.422 mmol) using the same method as described in example 43. The crude product was purified using silica gel column chromatography eluting with ethyl acetate:methanol:0.88 ammonia (100:0:0 to 95:5:0.5, by volume) to afford the title compound as a colourless oil, in 63% yield, 176 mg.

LCMS: ESI m/z 626 [M+H]⁺

¹H NMR (400 MHz, METHANOL-d₄) δ=1.26-1.34 (m, 10H), 1.46-1.59 (m, 4H), 1.63-1.71 (m, 2H), 1.85-1.93 (m, 2H), 2.33 (s, 3H), 2.39-2.43 (m, 4H), 2.53-2.57 (m, 2H), 2.69-2.77 (m, 2H), 3.70 (s, 2H), 4.60-4.66 (m, 1H), 6.77 (d, 1H), 7.18 (d, 1H), 7.23-7.44 (m, 8H), 7.55 (d, 1H) ppm.

### Example 55

### Biphenyl-2-yl-carbamic acid 1-{9-[(3,5-difluoro-2-hydroxy-benzyl)-methyl-amino]-nonyl}-piperidin-4-yl ester

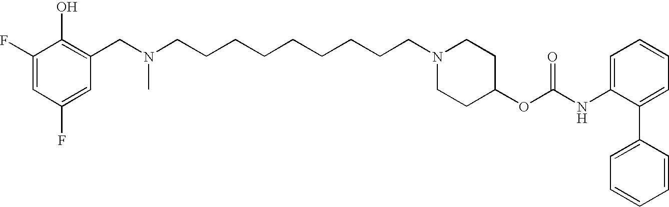

The title compound was prepared from biphenyl-2-yl-carbamic acid 1-(9-methylamino-nonyl)-piperidin-4-yl ester; dihydrochloride salt (Preparation 7, 40.0 mg, 89 μmol) and 3,5-difluoro-2-hydroxybenzaldehyde (14.1 mg, 89 μmol) using the same method as described in example 40.

LCMS Method A: RT 2.19 min (100% area)

ES m/z 594 [M+H]⁺.

### Example 56

### Biphenyl-2-yl-carbamic acid 1-{9-[(3,5-dichloro-2-hydroxy-benzoyl)-methyl-amino]-nonyl}-piperidin-4-yl ester

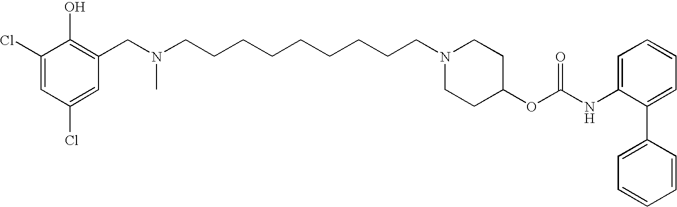

Biphenyl-2-yl-carbamic acid 1-(9-methylamino-nonyl)-piperidin-4-yl ester; dihydrochloride salt (Preparation 7, 40.2 mg, 89 μmol) was dissolved in dimethylformamide (0.5 ml) and added to a reaction vessel containing 3,5-dichloro-2-hydroxybenzoic acid (18.4 mg, 89 μmol). To the reaction mixture was then added a solution of 1-hydroxybenzotriazole monohydrate (16.4 mg, 107 μmol) in dimethylformamide (0.2 ml), a solution of (3-(dimethylamino)propyl)ethylcarbodimide hydrochloride (20.5 mg, 107 μmol) in dimethylformamide (0.4 ml) and diisopropylethylamine (38.7 μl, 222 μmol). The reaction was stirred at room temperature for 24 hours. The solvent was removed in vacuo and the residue partitioned between dichloromethane (2 ml) and water (2 ml). The aqueous phase was extracted with further dichloromethane (1 ml) and the combined organic layers were concentrated in vacuo. The crude residue was purified by HPLC method G to afford the title compound.

LCMS Method G: RT 2.98 min (100% area)

ES m/z 640 [M+H]⁺.

### Example 57

### Biphenyl-2-yl-carbamic acid 1-{9-[(3-chloro-2-hydroxy-benzoyl)-methyl-amino]-nonyl}-piperidin-4-yl ester

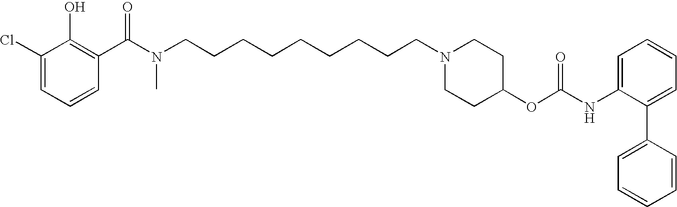

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 3-chloro-2-hydroxy benzoic acid (15.4 mg, 89 μmol) using the same method as described in example 56. The crude product was purified by HPLC method F and analysed by HPLC method G to yield the title compound.

LCMS Method G: RT 3.06 min (100% area)

ES m/z 606 [M+H]⁺.

### Example 58

### Biphenyl-2-yl-carbamic acid 1-{9-[(3,4-difluoro-2-hydroxy-benzoyl)-methyl-amino]-nonyl}-piperidin-4-yl ester

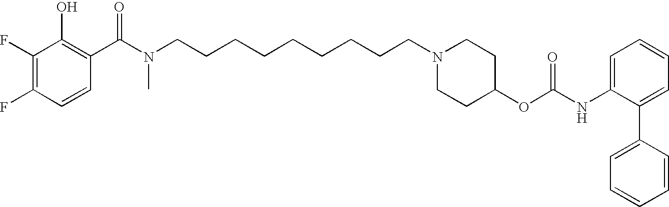

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 3,4-difluoro-2-hydroxy benzoic acid (15.5 mg, 89 μmol) using the same method as described in example 56. The crude product was purified by HPLC method F and analysed by HPLC method G to yield the title compound.

LCMS Method G: RT 2.87 min (100% area)

ES m/z 608 [M+H]⁺.

### Example 59

### Biphenyl-2-yl-carbamic acid 1-{9-[(3-fluoro-2-hydroxy-benzoyl)-methyl-amino]-nonyl}-piperidin-4-yl ester

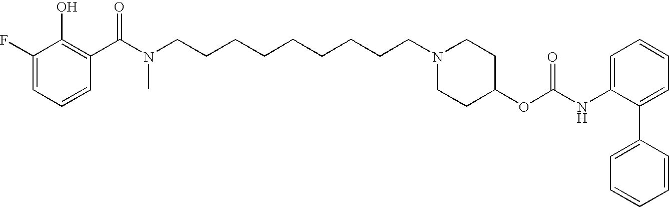

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 3-fluoro-2-hydroxy benzoic acid (13.9 mg, 89 μmol) using the same method as described in example 56. The crude product was purified by HPLC method F and analysed by HPLC method G to yield the title compound.

LCMS Method G: RT 3.13 min (100% area)

ES m/z 590 [M+H]⁺.

### Example 60

### Biphenyl-2-yl-carbamic acid 1-{9-[(4-chloro-2-hydroxy-benzoyl)-methyl-amino]-nonyl}-piperidin-4-yl ester

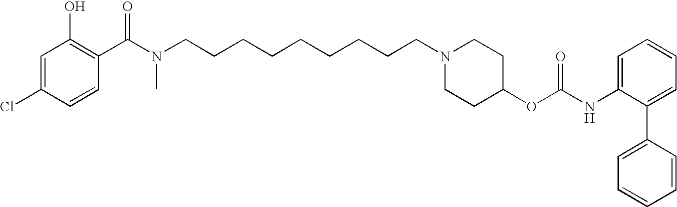

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 4-chloro-2-hydroxy benzoic acid (15.4 mg, 89 μmol) using the same method as described in example 56. The crude product was purified by HPLC method F and analysed by HPLC method G to yield the title compound.

LCMS Method G: RT 3.45 min (100% area)

ES m/z 606 [M+H]⁺.

### Example 61

### Biphenyl-2-yl-carbamic acid 1-{9-[(4-fluoro-2-hydroxy-benzoyl)-methyl-amino]-nonyl}-piperidin-4-yl ester

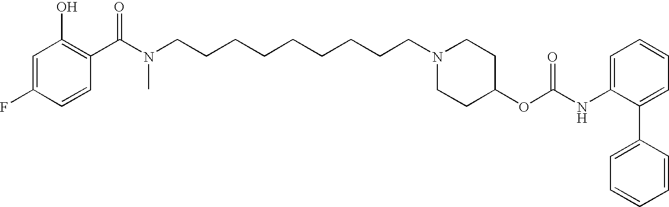

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 4-fluoro-2-hydroxy benzoic acid (13.9 mg, 89 μmol) using the same method as described in example 56. The crude product was purified by HPLC method F and analysed by HPLC method G to yield the title compound.

LCMS Method G: RT 3.47 min (100% area)

ES m/z 590 [M+H]⁺.

### Example 62

### Biphenyl-2-yl-carbamic acid 1-{9-[(4-chloro-3-hydroxy-benzoyl)-methyl-amino]-nonyl}-piperidin-4-yl ester

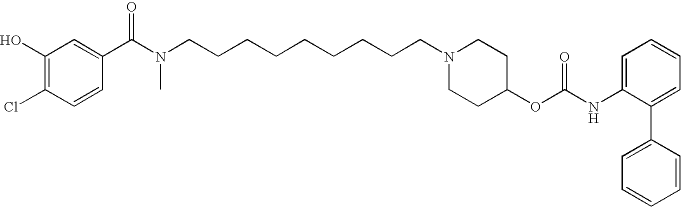

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 4-chloro-3-hydroxy benzoic acid (15.4 mg, 89 μmol) using the same method as described in example 56. The crude product was purified by HPLC method F and analysed by HPLC method G to yield the title compound.

LCMS Method G: RT 3.15 min (95% area)

ES m/z 606 [M+H]⁺.

### Example 63

### Biphenyl-2-yl-carbamic acid 1-{9-[(2-fluoro-4-hydroxy-benzoyl)-methyl-amino]-nonyl}-piperidin-4-yl ester

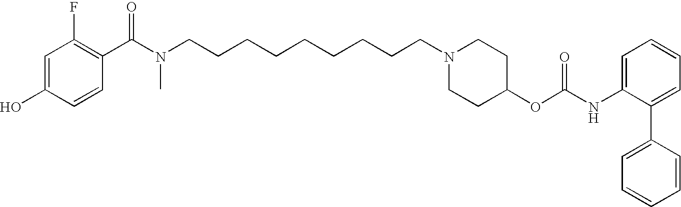

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 2-fluoro-4-hydroxy benzoic acid (13.9 mg, 89 μmol) using the same method as described in example 56. The crude product was purified by HPLC method F and analysed by HPLC method G to yield the title compound.

LCMS Method G: RT 3.32 min (94% area)

ES m/z 590 [M+H]⁺.

### Example 64

### Biphenyl-2-yl-carbamic acid 1-{9-[(2,3-difluoro-4-hydroxy-benzoyl)-methyl-amino]-nonyl}-piperidin-4-yl ester

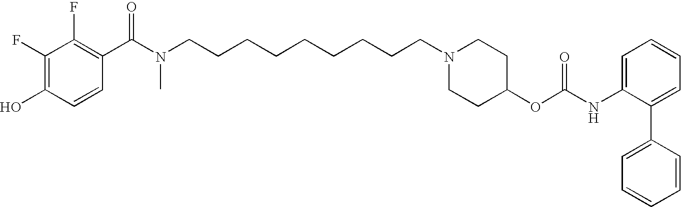

Biphenyl-2-yl-carbamic acid 1-(9-methylamino-nonyl)-piperidin-4-yl ester; dihydrochloride salt (Preparation 7, 2.5 g, 5.5 mmol) was dissolved in tetrahydrofuran (85 ml). To this solution was added 2,3-difluoro-4-hydroxy benzoic acid (1.16 g, 6.64 mmol), triethylamine (1.0 ml, 7.21 mmol), 4-dimethylaminopyridine (235 mg, 1.92 mmol) and (3-(dimethylamino)propyl)ethylcarbodimide hydrochloride (1.49 g, 7.75 mmol). The reaction mixture was stirred at room temperature for 15 minutes, then at 60° C. for 18 hours. The solvent was removed in vacuo and the residue partitioned between ethyl acetate (100 ml) and water (75 ml). The aqueous layer was further extracted with ethyl acetate (100 ml) and the combined organic layers were dried (magnesium sulphate) and concentrated in vacuo. The residue was purified using silica gel column chromatography eluting with dichloromethane:methanol:0.88 ammonia (100:0:0 to 95:5:0.5, by volume) to afford the title compound as a colourless oil, in 40% yield, 1.36 g.

LCMS: ESI m/z 608 [M+H]⁺

¹H NMR (400 MHz, METHANOL-d₄) δ=1.29-1.41 (m, 10H), 1.47-1.54 (m, 4H), 1.61-1.73 (m, 2H), 1.81-1.95 (m, 2H), 2.35-2.54 (m, 4H), 2.68-2.83 (m, 2H), 2.94-3.07 (m, 3H), 3.25-3.28 (m, 1H), 3.50-3.54 (m, 1H), 4.58-4.69 (m, 1H), 6.69-6.76 (m, 1H), 6.85-6.93 (m, 1H), 7.23-7.44 (m, 7H), 7.55 (d, 1H), 8.06 (d, 1H) ppm.

### Example 65

### Biphenyl-2-yl-carbamic acid 1-{9-[(2-chloro-4-hydroxy-benzoyl)-methyl-amino]-nonyl}-piperidin-4-yl ester

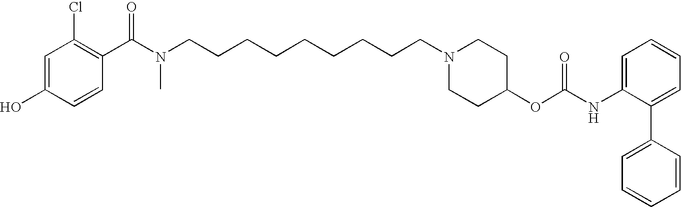

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 2-chloro-4-hydroxy benzoic acid hydrate (17.0 mg, 89 μmol) using the same method as described in example 56. The crude product was purified by HPLC method F and analysed by HPLC method G to yield the title compound.

LCMS Method G: RT 3.54 min (95% area)

ES m/z 606 [M+H]⁺.

### Example 66

### Biphenyl-2-yl-carbamic acid 1-(9-{[2-(3-hydroxy-phenyl)-acetyl]-methyl-amino}-nonyl)-piperidin-4-yl ester

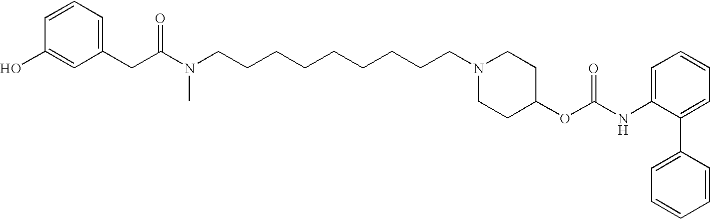

The title compound was prepared from biphenyl-2-yl-carbamic acid 1-(9-methylamino-nonyl)-piperidin-4-yl ester; dihydrochloride salt (Preparation 7, 2.50 g, 5.54 mmol) and 3-hydroxy phenyl acetic acid (1.01 g, 6.64 mmol) using the same method as described in example 64. The residue was purified using silica gel column chromatography eluting with dichloromethane:methanol:0.88 ammonia (100:0:0 to 90:10:1, by volume) to afford the title compound as a colourless oil, in 31% yield, 1.01 g.

100 mg of the title compound was suspended in heptane and slurried at 50° C. for 18 hours, and then at room temperature for 2 days. The solid was collected by filtration to yield the title compound as a white crystalline solid, 57 mg.

LCMS: ESI m/z 586 [M+H]⁺

¹H NMR (400 MHz, METHANOL-d₄) δ=1.22-1.33 (m, 10H), 1.36-1.54 (m, 4H), 1.58-1.67 (m, 2H), 1.80-1.88 (m, 2H), 2.24-2.35 (m, 4H), 2.63-2.70 (m, 2H), 2.90-2.99 (m, 3H), 3.33-3.39 (m, 2H), 3.66-3.68 (d, 2H), 4.57-4.62 (m, 1H), 6.64-6.71 (m, 3H), 7.09-7.13 (m, 1H), 7.23-7.44 (m, 8H), 7.55 (d, 1H) ppm.

### Example 67

### Biphenyl-2-yl-carbamic acid 1-{9-[(5-fluoro-2-hydroxy-benzoyl)-methyl-amino]-nonyl}-piperidin-4-yl ester

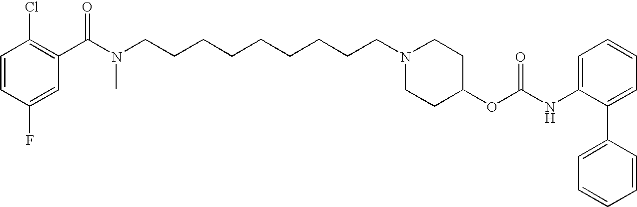

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 5-fluoro-2-hydroxy benzoic acid (13.9 mg, 89 μmol) using the same method as described in example 56. The crude product was purified by HPLC method F and analysed by HPLC method G to yield the title compound.

LCMS Method G: RT 3.95 min (100% area)

ES m/z 590 [M+H]⁺.

### Example 68

### Biphenyl-2-yl-carbamic acid 1-{9-[(5-chloro-2-hydroxy-benzoyl)-methyl-amino]-nonyl}-piperidin-4-yl ester

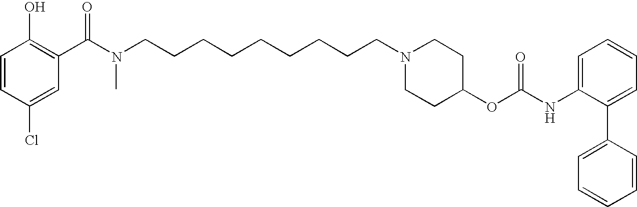

The title compound was prepared from biphenyl-2-yl-carbamic acid 1-(9-methylamino-nonyl)-piperidin-4-yl ester; dihydrochloride salt (Preparation 7, 40.2 mg, 89 μmol) and 5-chloro-2-hydroxy benzoic acid (15.4 mg, 89 μmol) using the same method as described in example 56. The crude product was purified by HPLC method F and analysed by HPLC method G to yield the title compound.

LCMS Method G: RT 3.78 min (100% area)

ES m/z 606 [M+H]⁺.

### Example 69

### Biphenyl-2-yl-carbamic acid 1-{9-[(3-fluoro-4-hydroxy-benzoyl)-methyl-amino]-nonyl}-piperidin-4-yl ester

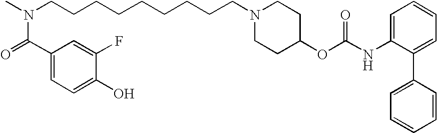

To a solution of biphenyl-2-yl-carbamic acid 1-(9-methylamino-nonyl)-piperidin-4-yl ester dihydrochloride salt (Preparation 7, 4.47 g, 9.90 mmol) in tetrahydrofuran (150 ml), was added 3-fluoro-4-hydroxybenzoic acid (1.85 g, 11.9 mmol), triethylamine (2.07 ml, 14.8 mmol), N,N-dimethylaminopyridine (484 mg, 3.96 mmol) and (3-(dimethylamino)propyl)ethyl carbodiimide hydrochloride (2.66 g, 13.9 mmol). The mixture was stirred at room temperature for 15 minutes and then at 60° C. for 18 hours. Further 3-fluoro-4-hydroxybenzoic acid (308 mg, 2.0 mmol) was added and the reaction heated at 60° C. for a further 18 hours. The solvent was removed in vacuo and residue partitioned between ethyl acetate (200 ml) and water (150 ml). The aqueous layer was further extracted with ethyl acetate (200 ml) and the combined organic layers dried over magnesium sulphate and concentrated in vacuo. The residue was dissolved in methanol/water (115 ml/23 ml), treated with potassium carbonate (12.9 g, 93.2 mmol) and heated at 50° C. for 18 hours. The solvent was removed in vacuo and residue partitioned between dichloromethane (200 ml) and water (200 ml). The organic layer was washed with brine (100 ml) and concentrated in vacuo. The residue was purified by column chromatography on silica gel eluting with dichloromethane:methanol:880 ammonia (100:0:0 to 95:5:0.5, by volume), to furnish the title compound as an oily foam, in 52% yield, 3.01 g.

LCMS: APCI ESI m/z 590 [M+H]⁺

¹H NMR (400 MHz, METHANOL-d₄) δ=1.12-1.40 (m, 10H), 1.45-1.54 (m, 2H), 1.56-1.68 (m, 4H), 1.81-1.91 (m, 2H), 2.29-2.40 (m, 4H), 2.64-2.75 (m, 2H), 3.01 (s, 3H), 3.34-3.53 (m, 2H), 4.58-4.65 (m, 1H), 6.92-6.96 (m, 1H), 7.05 (d, 1H), 7.12 (d, 1H), 7.23-7.44 (m, 8H), 7.55 (d, 1H) ppm.

### HPLC Methodology

### Method A:

| HPLC conditions | Analytical (QC) | | Preparative | |
|---|---|---|---|---|
| Column | 5 μm 4.6 × 50 mm | | Sunfire Prep C18 OBD | |
| | | | 5 μm 19 × 100 mm | |
| Temperature | Ambient | | Ambient | |
| Detection | UV 225 nm - ELSD-MS | | ELSD-MS | |
| System/Data file | | | | |
| Injection volume | 5 μL | | 1000 μL | |
| Flow rate | 1.5 mL/min | | 18 mL/min | |
| Mobile phase | | | A: H2O + 0.1% formic | |
| | | | acid | |
| | | | B: MeCN + 0.1% formic | |
| | | | acid | |
| | Time (min) | % B | Time (min) | % B |
| Gradient | 0 | 5 | 0-1.0 | 5 |
| | 0-3.0 | 5-95 | 1.0-7.0 | 5-98 |
| | 3.0-4.0 | 95 | 7.0-9.0 | 98 |
| | 4.0-4.1 | 95-5 | 9.0-9.10 | 98-5 |
| | 4.1-5.0 | 5 | 9.10-10 | 5 |

### Method D:

| HPLC conditions | Analytical (QC) | | Preparative | |
|---|---|---|---|---|
| Column | Sunfire C18 | | Gemini C18 | |
| | 5um 6 × 50 mm | | 10 * 150 mm 10 um | |
| Temperature | Ambient | | Ambient | |
| Detection | Diode Array/ELSD-MS | | MS and ELSD | |
| System/Data file | CTC - LC MS | | Fractionlynx 1 | |
| Injection volume | ~5 ml | | volume of each sample | |
| Flow rate | 1.5 mL/min | | 10 mL/min | |
| Mobile phase | A: H₂O + 0.1% formic | | A: H₂O + 0.2% formic | |
| | B: MeCN + 0.1% formic | | B: MeCN + 0.2% formic | |
| | acid | | acid | |
| | Time (min) | % B | Time (min) | % B |
| Gradient | 0 | 5 | initial | 20 |
| | 0-3.0 | 5-95 | 1 | 20 |
| | 3.0-4.0 | 95 | 5.4 | 70 |
| | 4.0-4.1 | 95-5 | 6.33 | 98 |
| | 4.1-5.0 | 5 | 6.4 | 20 |
| | | | 7 | 20 |

### Method F:

| HPLC conditions | Analytical (QC) | | Preparative | |
|---|---|---|---|---|
| Column | 5 μm 4.6 × 50 mm | | Sunfire Prep C18 OBD | |
| | | | 5 μm 19 × 100 mm | |
| Temperature | Ambient | | Ambient | |
| Detection | UV 225 nm - ELSD-MS | | ELSD-MS | |
| System/Data file | CTC - MUX1 | | Fractionlynx 4 | |
| Injection volume | 5 μL | | 1000 μL | |
| Flow rate | 1.5 mL/min | | 10 mL/min | |
| Mobile phase | | | A: H₂O + 0.1% formic acid | |
| | | | B: MeCN + 0.1% formic | |
| | | | acid | |
| | Time (min) | % B | Time (min) | % B |
| Gradient | 0 | 5 | 0-1.0 | 5 |
| | 0-3.0 | 5-95 | 1.0-7.0 | 5-98 |
| | 3.0-4.0 | 95 | 7.0-9.0 | 98 |
| | 4.0-4.1 | 95-5 | 9.0-9.10 | 98-5 |
| | 4.1-5.0 | 5 | 9.10-10 | 5 |

### Method G:

| HPLC conditions | Analytical (QC) | | Preparative | |
|---|---|---|---|---|
| Column | XTerra C18 | | XTerra Prep C18 | |
| | 5 μm 4.6 × 50 mm | | 5 μm 19 × 100 mm | |
| Temperature | Ambient | | Ambient | |
| Detection | UV 225 nm - ELSD-MS | | ELSD-MS | |
| System/Data file | CTC - MUX1 | | Fractionlynx 2 | |
| Injection volume | 5 μL | | 1000 μL | |
| Flow rate | 1.5 mL/min | | 18 mL/min | |
| Mobile phase | A: H₂O + 0.1% | | A: H₂O + 0.1% | |
| | ammonia | | diethylamine | |
| | B: MeCN + 0.1% | | B: MeCN + 0.1% | |
| | ammonia | | diethylamine | |
| | Time (min) | % B | Time (min) | % B |
| Gradient | 0 | 5 | 0-1.0 | 5 |
| | 0-3.0 | 5-95 | 1.0-7.0 | 5-98 |
| | 3.0-4.0 | 95 | 7.0-9.0 | 98 |
| | 4.0-4.1 | 95-5 | 9.0-9.10 | 98-5 |
| | 4.1-5.0 | 5 | 9.10-10 | 5 |

### Cell BASED POTENCY ASSESSMENT at the Human Recombinant M₃ Muscarinic Receptor

M₃ potency was determined in CHO-K1 cells transfected with the NFAT-Betalactamase gene. CHO (Chinese Hamster Ovary) cells recombinantly expressing the human muscarinic M₃ receptor were transfected with the NFAT_β-Lac_Zeo plasmid. Cells were grown in DMEM with Glutamax-1, supplemented with 25 mM HEPES (Life Technologies 32430-027), containing 10% FCS (Foetal Calf Serum; Sigma F-7524), 1 nM Sodium pyruvate (Sigma S-8636), NEAA (non-Essential Amino Acids; Invitrogen 11140-035) and 200 μg/ml Zeocin (Invitrogen R250-01).

hM₃ β-Lactamase Assay Protocol

Cells were harvested for assay when they reached 80-90% confluency using enzyme free cell Dissociation Solution (Life technologies 13151-014) incubated with the cells for 5 min at 37° C. in an atmosphere containing 5% CO₂. Detached cells were collected in warmed growth media and centrifuged at 2000 rpm for 10 min, washed in PBS (Phosphate Buffered Saline; Life Technologies 14190-094) and centrifuged again as just described. The cells were re-suspended at 2×10⁵ cells/ml in growth medium (composition as described above). 20 μl of this cell suspension was added to each well of a 384 well black clear bottomed plate (Greiner Bio One 781091-PFI). The assay buffer used was PBS supplemented with 0.05% Pluronic F-127 (Sigma 9003-11-6) and 2.5% DMSO. Muscarinic M₃ receptor signalling was stimulated using 80 nM carbamyl choline (Aldrich N240-9) incubated with the cells for 4 h at 37° C./5% CO₂ and monitored at the end of the incubation period using a Tecan SpectraFluor+plate reader (λ−excitation 405 nm, emission 450 nm and 503 nm). M₃ receptor antagonists under test were added to the assay at the beginning of the 4 h incubation period and compound activity measured as the concentration dependent inhibition of the carbamyl choline induced signal. Inhibition curves were plotted and IC₅₀ values generated using a 4-parameter sigmoid fit and converted to Ki values using the Cheng-Prusoff correction and the K_{D} value for carbamyl choline in the assay.

### Binding Affinity Assessment at the Human Recombinant M₃ Muscarinic Receptor

### Membrane Preparation

Cell Pellets from CHO (Chinese Hamster Ovary) cells recombinantly expressing the human muscarinic M₃ receptor were homogenised in 20 mM HEPES (pH7.4) and centrifuged at 48000×g for 20 min at 4° C. The pellet was re-suspended in buffer and the homogenisation and centrifugation steps repeated. The resulting pellet was re-suspended in 1 ml buffer per 1 ml original packed cell volume and the homogenisation step repeated. Protein estimation was carried out on the suspension and 1 ml aliquots of ˜1 mg/ml frozen at −80° C.

hM₃ Competition Binding Assay Protocol

Membranes (5 □g/well) were incubated with ³H-NMS (at a concentration 5×K_{D}) plus/minus test compound for 24 hr at RT (room temperature) in a 1 ml polystyrene 96-well deep well block. The final assay volume was 200 □l, comprising of: 20 □l plus/minus test compound; 20 □l ³H-NMS (Perkin Elmer NEN 636) and 160 □l membrane solution. Total Binding was defined with 0.1% DMSO; Non-Specific Binding was defined with 1 □M Atropine. Assay buffer was 20 mM Hepes (pH 7.4).

Once all assay components were added, plates were covered and incubated at room temperature for 24 hrs with shaking. The assay was terminated by rapidly filtering through GF/B Unifilter plates pre-soaked with 0.5% polyethylenimine, using a Packard filtermate harvester, the filter plate was then washed with 3×1 ml 4° C. assay buffer. The filter plates were dried at 45° C. for 1 hour. The bottoms of the filter plates were sealed and 50 □l/well of Microscint ‘0’ added, the top of the plates were sealed with a Topseal. Following 90 mins, the plates were read on an NXT Topcount (1 minute read time per well).

The resulting data was expressed as a percentage of the specific binding (Specific binding=Total binding−Non-Specific Binding). % specific binding versus test compound concentration was plotted to determine an IC₅₀ from a sigmoid curve using an in-house data analysis programme. IC₅₀ values corrected to Ki values by applying the Cheng-Prussoff equation:

Cheng-Prussoff equation:

$${K}_{i} = \frac{{IC}_{50}}{1 + [ L ] / {K}_{D}}$$

where IC₅₀ is the concentration of unlabelled drug which inhibits by 50% the specific radioligand binding. [L] is the free radioligand concentrations and K_{D} and Kᵢ are the equilibrium dissociation constants of the radioligand and unlabelled drug respectively.

It has thus been found that compounds of formula (I) according to the present invention that have been tested in the above assays show hM₃ receptor antagonist activity as listed in the table below:

| | CHO cell β-lactamase | CHO cell binding assay |
|---|---|---|
| | hM₃ Ki (nM) | hM₃ Ki (nM) |
| 1 | 0.0697 | 1.60 |
| 2 | 0.840 | 1.05 |
| 3 | 0.779 | 2.33 |
| 4 | 0.0415 | 1.19 |
| 5 | 0.894 | 0.521 |
| 6 | 0.222 | 0.778 |
| 7 | 1.36 | 0.997 |
| 8 | 2.68 | 4.81 |
| 9 | 8.81 | n.d. |
| 10 | 0.803 | 1.07 |
| 11 | 8.15 | 2.24 |
| 12 | 7.32 | n.d. |
| 13 | 18.0 | n.d. |
| 14 | 13.9 | n.d. |
| 15 | 0.396 | 2.37 |
| 16 | 0.389 | 2.98 |
| 17 | 0.250 | 2.15 |
| 18 | 0.353 | 2.29 |
| 19 | 1.32 | 1.45 |
| 20 | 0.918 | 1.95 |
| 21 | 0.682 | 1.26 |
| 22 | 0.149 | 0.865 |
| 23 | 0.276 | 2.38 |
| 24 | 17.0 | n.d. |
| 25 | 0.854 | 4.34 |
| 26 | 4.51 | n.d. |
| 27 | 0.208 | 1.10 |
| 28 | 3.59 | 3.83 |
| 29 | 0.816 | 1.35 |
| 30 | 0.705 | 1.31 |
| 31 | 0.728 | 2.47 |
| 32 | 1.34 | 3.04 |
| 33 | 2.72 | 6.04 |
| 34 | 0.960 | 0.787 |
| 35 | 1.64 | 1.38 |
| 36 | 2.24 | 0.235 |
| 37 | 1.12 | 0.342 |
| 38 | 1.48 | 0.175 |
| 39 | n.d. | 1.13 |
| 40 | n.d. | 0.638 |
| 41 | n.d. | 1.3 |
| 42 | n.d. | 0.233 |
| 43 | n.d. | 0.295 |
| 44 | n.d. | 0.212 |
| 45 | n.d. | 0.212 |
| 46 | n.d. | 0.755 |
| 47 | n.d. | 0.529 |
| 48 | n.d. | 0.182 |
| 49 | n.d. | 0.285 |
| 50 | n.d. | 0.22 |
| 51 | n.d. | 0.231 |
| 52 | n.d. | 0.637 |
| 53 | n.d. | 0.345 |
| 54 | n.d. | 0.233 |
| 55 | n.d. | 0.417 |
| 56 | n.d. | 11.3 |
| 57 | n.d. | 0.87 |
| 58 | n.d. | 1.83 |
| 59 | n.d. | 1.27 |
| 60 | n.d. | 3.61 |
| 61 | n.d. | 0.778 |
| 62 | n.d. | 1.21 |
| 63 | n.d. | 1.12 |
| 64 | n.d. | 2.00 |
| 65 | n.d. | 0.628 |
| 66 | n.d. | 0.292 |
| 67 | n.d. | 0.828 |
| 68 | n.d. | 2.27 |
| 69 | n.d. | 0.615 |

n.d.—not determined

### Guinea Pig Trachea Assay

Male, Dunkin-Hartley guinea-pigs weighing 350-450 g are culled in a rising concentration of CO₂, followed by exsanguinations of the vena cava. Tracheas are dissected from the larynx to the entry point into the chest cavity and then placed in fresh, oxygenated, modified Krebs buffer solution (Krebs containing 10 μM propranolol, 10 μM guanethidine and 3 μM indomethacin) at room temperature. The tracheas are opened by cutting through the cartilage opposite the trachealis muscle. Strips approximately 3-5 cartilage rings wide are cut. A cotton thread is attached to the cartilage at one end of the strip for attachment to the force transducer and a cotton loop made at the other end to anchor the tissue in the organ bath. The strips are mounted in 5 ml organ baths filled with warm (37° C.) aerated modified Krebs. The pump flow rate is set to 1.0 ml/min and the tissues washed continuously. Tissues are placed under an initial tension of 1000 mg. Tissues are re-tensioned after 15 and 30 minutes, then allowed to equilibrate for a further 30-45 minutes.

Tissues are subjected to electrical field stimulation (EFS) of the following parameters: 10 s trains every 2 minutes, 0.1 ms pulse width, 10 Hz and 10-30V. The voltage is raised 5V every 10 min within the stated range until a maximum contractile response for each tissue is observed. This just maximum voltage for each tissue is then used throughout the remainder of the experiment. Following equilibration to EFS for 20 min, the pump is stopped, and after 15 min control readings are taken over a 8-10 min period (4-5 responses). Compound is then added to each tissue as a bolus dose at 30×Ki (determined at the human M₃ receptor expressed in CHO cells in a filtration binding assay), and left to incubate for 2 h. Compound is then washed from tissues using a rapid wash with modified Krebs for 1 min and flow is restored to 1 ml/min for the remainder of the experiment. At the end of the experiment tissues are challenged with histamine (1 □M) to determine viability. Readings taken during the experiment are automatically collected using Notocord® software. The raw data are converted into percent response taking into account measurements of inhibition of the EFS response. After starting washout, the times taken for the tissue to recover by 25% from the inhibition induced are recorded and used as a measure of compound duration of action. Tissue viability limits the duration of the experiment to 16 h post-compound washout. Compounds are typically tested at n=2 to 5 to estimate duration of action.

Alternatively the following Guinea Pig Trachea assay can also be used:

Trachea were removed from male Dunkin-Hartley guinea-pigs (wt 350-450 g) and following removal of adherent connective tissue, an incision was made through the cartilage opposite the trachealis muscle and tracheal strips 3-5 cartilage rings wide prepared. The tracheal strips were suspended between an isometric strain gauge and a fixed tissue hook with the muscle in the horizontal plane in 5 ml tissue baths under an initial tension of 1 g and bathed in warmed (37° C.) aerated (95% O₂/5% CO₂) Krebs solution containing 3 μM indomethacin and 10 μM guanethidine. The tissues were positioned between parallel platinum wire electrodes (˜1 cm gap). A constant 1 ml/min flow of fresh Krebs solution (of the above composition) was maintained through the tissue baths using peristaltic pumps. The tissues were allowed to equilibrate for an hour with re-tensioning to 1 g at 15 min and 30 min from the start of the equilibration period. At the end of the equilibration, tissues were electrically field stimulated (EFS) using the following parameters: 10V, 10 Hz 0.1 ms pulse width with 10 sec trains every 2 min. In each tissue a voltage response curve was constructed over the range 10 v-30V (keeping all other stimulation parameters constant) to determine a just maximal stimulation. Using these stimulation parameters EFS responses were 100% nerve mediated and 100% cholinergic as confirmed by blockade by 1 μM tetrodotoxin or 1 μM atropine. Tissues were then repeatedly stimulated at 2 min intervals until the responses were reproducible. The peristaltic pump was stopped 20 min prior to the addition of the study compound and the average twitch contraction over the last 10 min recorded as the control response. The study compound was added to the tissue baths, with each tissue receiving a single concentration of compound and allowed to equilibrate for 2 h. At 2 h post addition the inhibition of the EFS response was recorded and IC₅₀ curves generated using a range of compound concentrations over tracheal strips from the same animal. The tissues were then rapidly washed and the 1 ml/min perfusion with Krebs solution re-established. Tissues were stimulated for a further 16 h and recovery of the EFS response recorded. At the end of the 16 h, 10 μM histamine was added to the baths to confirm tissue viability. The just max concentration (tested concentration giving a response >70% inhibition but less than 100%) of antagonist was identified from the IC₅₀ curve and the time to 25% recovery of the induced inhibition (T₂₅) calculated in tissues receiving this concentration. Compounds are typically tested at n=2 to 5 to estimate duration of action.

### Assessment of Drug-Drug Interaction Potential

As already stated hereinbefore, the potential for drug-drug interaction of a compound can be simulated with commercially available software, for example Simcyp® using in vitro metabolism data that can easily be generated by following the user's guideline of the Simcyp® software and according to standard protocols that are well-known to the skilled person. As a matter of example, glucuronidation data may be determined using a methodology similar to the reference methodology described in Kilford et al., Drug metabolism and Disposition, Vol. 37, No. 1, pp. 82-89.

1. A compound of formula (I): 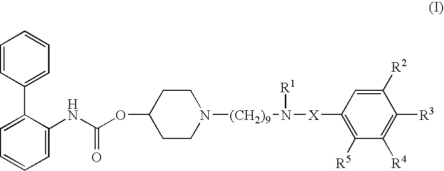
or a pharmaceutically acceptable salt thereof, wherein:
X is —C(═O)CH₂—, or —C(═O)—; and R¹ is H or methyl; or alternatively
X is —CH₂— and R¹ is H, methyl or a group of formula: 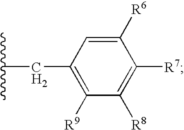
wherein one of R⁶, R⁷, R⁸ and R⁹ is hydroxy, one of R⁶, R⁷, R⁸ and R⁹ is halo, one of R⁶, R⁷, R⁸ and R⁹ is H, and one of R⁶, R⁷, R⁸ and R⁹ is selected from H or halo;
one of R², R³, R⁴ and R⁵ is hydroxyl, one of R², R³, R⁴ and R⁵ is H, one of R², R³, R⁴ and R⁵ is halo, and
one of R², R³, R⁴ and R⁵ is H or halo, or alternatively when X is —C(═O)CH₂— and R¹ is methyl then R⁴ can also be hydroxyl while R², R³ and R⁵ are H.

2. A compound according to claim 1, or a pharmaceutically acceptable salt thereof, which is:
Biphenyl-2-yl-carbamic acid 1-[9-(3-chloro-4-hydroxy-benzoylamino)-nonyl]-piperidin-4-yl ester;
Biphenyl-2-yl-carbamic acid 1-[9-(2-fluoro-3-hydroxy-benzylamino)-nonyl]-piperidin-4-yl ester;
Biphenyl-2-yl-carbamic acid 1-[9-(3-chloro-5-fluoro-2-hydroxy-benzylamino)-nonyl]-piperidin-4-yl ester;
Biphenyl-2-yl-carbamic acid 1-{9-[2-(3-chloro-4-hydroxy-phenyl)-acetylamino]-nonyl}-piperidin-4-yl ester;
Biphenyl-2-yl-carbamic acid 1-[9-(2-chloro-3-hydroxy-benzylamino)-nonyl]-piperidin-4-yl ester;
Biphenyl-2-yl-carbamic acid 1-[9-(3-fluoro-4-hydroxy-benzoylamino)-nonyl]-piperidin-4-yl ester;
Biphenyl-2-yl-carbamic acid 1-[9-(4-fluoro-3-hydroxy-benzoylamino)-nonyl]-piperidin-4-yl ester;
Biphenyl-2-yl-carbamic acid 1-[9-(3-fluoro-2-hydroxy-benzoylamino)-nonyl]-piperidin-4-yl ester;
Biphenyl-2-yl-carbamic acid 1-[9-(5-fluoro-2-hydroxy-benzoylamino)-nonyl]-piperidin-4-yl ester;
Biphenyl-2-yl-carbamic acid 1-[9-(3,5-dichloro-2-hydroxy-benzoylamino)-nonyl]-piperidin-4-yl ester;
Biphenyl-2-yl-carbamic acid 1-[9-(4-chloro-2-hydroxy-benzoylamino)-nonyl]-piperidin-4-yl ester;
Biphenyl-2-yl-carbamic acid 1-(9-{[2-(3-chloro-4-hydroxy-phenyl)-acetyl]-methyl-amino}-nonyl)-piperidin-4-yl ester;
Biphenyl-2-yl-carbamic acid 1-{9-[(4-fluoro-3-hydroxy-benzoyl)-methyl-amino]-nonyl}-piperidin-4-yl ester;
Biphenyl-2-yl-carbamic acid 1-{9-[(3-chloro-4-hydroxy-benzoyl)-methyl-amino]-nonyl}-piperidin-4-yl ester;
Biphenyl-2-yl-carbamic acid 1-(9-{[2-(3-fluoro-4-hydroxy-phenyl)-acetyl]-methyl-amino}-nonyl)-piperidin-4-yl ester;
Biphenyl-2-yl-carbamic acid 1-[9-(4-chloro-3-hydroxy-benzoylamino)-nonyl]-piperidin-4-yl ester;
Biphenyl-2-yl-carbamic acid 1-[9-(2-fluoro-4-hydroxy-benzoylamino)-nonyl]-piperidin-4-yl ester;
Biphenyl-2-yl-carbamic acid 1-[9-(3,5-dichloro-4-hydroxy-benzylamino)-nonyl]-piperidin-4-yl ester;
Biphenyl-2-yl-carbamic acid 1-[9-(3,5-dichloro-4-hydroxy-benzylamino)-nonyl]-piperidin-4-yl ester;
naphthalene-1,5-disulfonate salt;
Biphenyl-2-yl-carbamic acid 1-[9-(2,3-difluoro-4-hydroxy-benzoylamino)-nonyl]-piperidin-4-yl ester;
Biphenyl-2-yl-carbamic acid 1-[9-(2-hydroxy-5-chloro-benzoylamino)-nonyl]-piperidin-4-yl ester;
Biphenyl-2-yl-carbamic acid 1-[9-(4-hydroxy-3,5-dichloro-benzoylamino)-nonyl]-piperidin-4-yl ester;
Biphenyl-2-yl-carbamic acid 1-[9-(2-hydroxy-4-fluoro-benzoylamino)-nonyl]-piperidin-4-yl ester;
Biphenyl-2-yl-carbamic acid 1-[9-(3,5-difluoro-4-hydroxy-benzylamino)-nonyl]-piperidin-4-yl ester;
Biphenyl-2-yl-carbamic acid 1-[9-(2-hydroxy-4,5-dichloro-benzylamino)-nonyl]-piperidin-4-yl ester;
Biphenyl-2-yl-carbamic acid 1-[9-(2-hydroxy-3,5-difluoro-benzylamino)-nonyl]-piperidin-4-yl ester;
Biphenyl-2-yl-carbamic acid 1-[9-(2-hydroxy-3-fluoro-benzylamino)-nonyl]-piperidin-4-yl ester;
Biphenyl-2-yl-carbamic acid 1-[9-(2-hydroxy-3,5-dichloro-benzylamino)-nonyl]-piperidin-4-yl ester;
Biphenyl-2-yl-carbamic acid 1-[9-(5-chloro-2-hydroxy-benzylamino)-nonyl]-piperidin-4-yl ester;
Biphenyl-2-yl-carbamic acid 1-[9-(2-hydroxy-4-chloro-5-fluoro-benzylamino)-nonyl]-piperidin-4-yl ester;
Biphenyl-2-yl-carbamic acid 1-[9-(3-chloro-4-hydroxy-benzylamino)-nonyl]-piperidin-4-yl ester;
Biphenyl-2-yl-carbamic acid 1-[9-(2-hydroxy-5-fluoro-benzylamino)-nonyl]-piperidin-4-yl ester;
Biphenyl-2-yl-carbamic acid 1-[9-(3-hydroxy-4-fluoro-benzylamino)-nonyl]-piperidin-4-yl ester;
Biphenyl-2-yl-carbamic acid 1-[9-(2,4-dichloro-3-hydroxy-benzylamino)-nonyl]-piperidin-4-yl ester;
Biphenyl-2-yl-carbamic acid 1-[9-(4-hydroxy-3-fluoro-benzylamino)-nonyl]-piperidin-4-yl ester;
Biphenyl-2-yl-carbamic acid 1-{9-[bis-(2-chloro-3-hydroxy-benzyl)-amino]-nonyl}-piperidin-4-yl ester;
Biphenyl-2-yl-carbamic acid 1-{9-[(3-fluoro-2-hydroxy-benzyl)-methyl-amino]-nonyl}-piperidin-4-yl ester;
Biphenyl-2-yl-carbamic acid 1-{9-[(4,5-dichloro-2-hydroxy-benzyl)-methyl-amino]-nonyl}-piperidin-4-yl ester;
Biphenyl-2-yl-carbamic acid 1-{9-[(4-fluoro-3-hydroxy-benzyl)-methyl-amino]-nonyl}-piperidin-4-yl ester;
Biphenyl-2-yl-carbamic acid 1-{9-[(4-chloro-5-fluoro-2-hydroxy-benzyl)-methyl-amino]-nonyl}-piperidin-4-yl ester;
Biphenyl-2-yl-carbamic acid 1-{9-[(3-chloro-4-hydroxy-benzyl)-methyl-amino]-nonyl}-piperidin-4-yl ester;
Biphenyl-2-yl-carbamic acid 1-{9-[(3-fluoro-4-hydroxy-benzyl)-methyl-amino]-nonyl}-piperidin-4-yl ester;
Biphenyl-2-yl-carbamic acid 1-{9-[(5-chloro-2-hydroxy-benzyl)-methyl-amino]-nonyl}-piperidin-4-yl ester;
Biphenyl-2-yl-carbamic acid 1-{9-[(2-chloro-3-hydroxy-benzyl)-methyl-amino]-nonyl}-piperidin-4-yl ester;
Biphenyl-2-yl-carbamic acid 1-{9-[(3,5-dichloro-4-hydroxy-benzyl)-methyl-amino]-nonyl}-piperidin-4-yl ester;
Biphenyl-2-yl-carbamic acid 1-{9-[(2-fluoro-3-hydroxy-benzyl)-methyl-amino]-nonyl}-piperidin-4-yl ester;
Biphenyl-2-yl-carbamic acid 1-{9-[(3,5-difluoro-4-hydroxy-benzyl)-methyl-amino]-nonyl}-piperidin-4-yl ester;
Biphenyl-2-yl-carbamic acid 1-{9-[(2,4-dichloro-3-hydroxy-benzyl)-methyl-amino]-nonyl}-piperidin-4-yl ester;
Biphenyl-2-yl-carbamic acid 1-{9-[(3,5-difluoro-2-hydroxy-benzyl)-methyl-amino]-nonyl}-piperidin-4-yl ester;
Biphenyl-2-yl-carbamic acid 1-{9-[(3,5-dichloro-2-hydroxy-benzoyl)-methyl-amino]-nonyl}-piperidin-4-yl ester;
Biphenyl-2-yl-carbamic acid 1-{9-[(3-chloro-2-hydroxy-benzoyl)-methyl-amino]-nonyl}-piperidin-4-yl ester;
Biphenyl-2-yl-carbamic acid 1-{9-[(3,4-difluoro-2-hydroxy-benzoyl)-methyl-amino]-nonyl}-piperidin-4-yl ester;
Biphenyl-2-yl-carbamic acid 1-{9-[(3-fluoro-2-hydroxy-benzoyl)-methyl-amino]-nonyl}-piperidin-4-yl ester;
Biphenyl-2-yl-carbamic acid 1-{9-[(4-chloro-2-hydroxy-benzoyl)-methyl-amino]-nonyl}-piperidin-4-yl ester;
Biphenyl-2-yl-carbamic acid 1-{9-[(4-fluoro-2-hydroxy-benzoyl)-methyl-amino]-nonyl}-piperidin-4-yl ester;
Biphenyl-2-yl-carbamic acid 1-{9-[(4-chloro-3-hydroxy-benzoyl)-methyl-amino]-nonyl}-piperidin-4-yl ester;
Biphenyl-2-yl-carbamic acid 1-{9-[(2,3-difluoro-4-hydroxy-benzoyl)-methyl-amino]-nonyl}-piperidin-4-yl ester;
Biphenyl-2-yl-carbamic acid 1-(9-{[2-(3-hydroxy-phenyl)-acetyl]-methyl-amino}-nonyl)-piperidin-4-yl ester;
Biphenyl-2-yl-carbamic acid 1-{9-[(5-fluoro-2-hydroxy-benzoyl)-methyl-amino]-nonyl}-piperidin-4-yl ester;
Biphenyl-2-yl-carbamic acid 1-{9-[(5-chloro-2-hydroxy-benzoyl)-methyl-amino]-nonyl}-piperidin-4-yl ester;
or
Biphenyl-2-yl-carbamic acid 1-{9-[(3-fluoro-4-hydroxy-benzoyl)-methyl-amino]-nonyl}-piperidin-4-yl ester.

3. A compound according to claim 1, or a pharmaceutically acceptable salt thereof, which is:
Biphenyl-2-yl-carbamic acid 1-(9-{[2-(3-chloro-4-hydroxy-phenyl)-acetyl]-methyl-amino}-nonyl)-piperidin-4-yl ester;
Biphenyl-2-yl-carbamic acid 1-{9-[(3-chloro-4-hydroxy-benzoyl)-methyl-amino]-nonyl}-piperidin-4-yl ester;
Biphenyl-2-yl-carbamic acid 1-[9-(2,4-dichloro-3-hydroxy-benzylamino)-nonyl]-piperidin-4-yl ester;
Biphenyl-2-yl-carbamic acid 1-{9-[(2-chloro-3-hydroxy-benzyl)-methyl-amino]-nonyl}-piperidin-4-yl ester;
Biphenyl-2-yl-carbamic acid 1-{9-[(2,3-difluoro-4-hydroxy-benzoyl)-methyl-amino]-nonyl}-piperidin-4-yl ester;
Biphenyl-2-yl-carbamic acid 1-(9-{[2-(3-hydroxy-phenyl)-acetyl]-methyl-amino}-nonyl)-piperidin-4-yl ester;
or
Biphenyl-2-yl-carbamic acid 1-{9-[(3-fluoro-4-hydroxy-benzoyl)-methyl-amino]-nonyl}-piperidin-4-yl ester.

4. Biphenyl-2-yl-carbamic acid 1-(9-{[2-(3-hydroxy-phenyl)-acetyl]-methyl-amino}-nonyl)-piperidin-4-yl ester or a pharmaceutically acceptable salt thereof.

5. Biphenyl-2-yl-carbamic acid 1-{9-[(3-fluoro-4-hydroxy-benzoyl)-methyl-amino]-nonyl}-piperidin-4-yl ester or a pharmaceutically acceptable salt thereof.

6. A pharmaceutical composition comprising a compound of claim 1 or a pharmaceutically acceptable salt thereof and a pharmaceutically acceptable excipient.

7. A method of treating a disease, disorder or condition in a mammal in need thereof, said method comprising administering to said mammal a therapeutically effective amount of a compound of claim 1, or a pharmaceutically acceptable salt thereof, wherein said disease, disorder or condition is asthma, chronic or acute bronchoconstriction, bronchitis, small airways obstruction, emphysema, obstructive or inflammatory airways disease, acute lung injury or bronchiectasis.

8. A method according to claim 7 wherein said asthma is selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis.

9. A method according to claim 7 wherein said obstructive or inflammatory airways disease is selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension.

10. A method according to claim 7 wherein said bronchitis is selected from the group consisting of chronic bronchitis, acute bronchitis, acute laryngotracheal bronchitis, arachidic bronchitis, catarrhal bronchitis, croupus bronchitis, dry bronchitis, infectious asthmatic bronchitis, productive bronchitis, staphylococcus bronchitis, streptococcal bronchitis and vesicular bronchitis.

11. A method according to claim 7 wherein said bronchiectasis is selected from the group consisting of cylindric bronchiectasis, sacculated bronchiectasis, fusiform bronchiectasis, capillary bronchiectasis, cystic bronchiectasis, dry bronchiectasis and follicular bronchiectasis.

12. The method according to claim 7 wherein said disease, disorder or condition is asthma or chronic obstructive pulmonary disease (COPD).

13. A method of treating a disease, disorder or condition in a mammal in need thereof, said method comprising administering to said mammal a therapeutically effective amount of a compound of claim 1, or a pharmaceutically acceptable salt thereof, wherein said disease, disorder or condition is inflammatory bowel disease, irritable bowel disease, diverticular disease, motion sickness, gastric ulcers, radiological examination of the bowel, symptomatic treatment of BPH (benign prostatic hyperplasia), NSAID induced gastric ulceration, urinary incontinence (including urgency, frequency, urge incontinence, overactive bladder, nocturia and Lower urinary tract symptoms), cycloplegia, mydriatics or Parkinson's disease.

14. A pharmaceutical composition comprising a compound of claim 1 or a pharmaceutically acceptable salt thereof and another therapeutic agent selected from:
(a) 5-Lipoxygenase (5-LO) inhibitors or 5-lipoxygenase activating protein (FLAP) antagonists;
(b) Leukotriene antagonists (LTRAs) including antagonists of LTB₄, LTC₄, LTD₄, and LTE₄;
(c) Histamine receptor antagonists including H1 and H3 antagonists;
(d) α₁- and α₂-adrenoceptor agonist vasoconstrictor sympathomimetic agents for decongestant use;
(e) PDE inhibitors including PDE3, PDE4 and PDE5 inhibitors;
(f) Beta 2 receptor agonists;
(g) Theophylline;
(h) Sodium cromoglycate;
(i) COX inhibitors both non-selective and selective COX-1 or COX-2 inhibitors (NSAIDs);
(j) Prostaglandin receptor antagonists and inhibitors of prostaglandin synthase;
(k) Oral and inhaled glucocorticosteroids;
(l) Dissociated agonists of the corticoid receptor (DAGR);
(m) Monoclonal antibodies active against endogenous inflammatory entities;
(n) Anti-tumor necrosis factor (anti-TNF-α) agents;
(o) Adhesion molecule inhibitors including VLA-4 antagonists;
(p) Kinin-B₁- and B₂-receptor antagonists;
(q) Immunosuppressive agents including inhibitors of the IgE pathway and cyclosporine;
(r) Inhibitors of matrix metalloproteases (MMPs);
(s) Tachykinin NK₁, NK₂ and NK₃ receptor antagonists;
(t) Protease inhibitors such as elastase inhibitors;
(u) Adenosine A2a receptor agonists and A2b antagonists;
(v) Inhibitors of urokinase;
(w) Compounds that act on dopamine receptors such as D2 agonists;
(x) Modulators of the NFκβ pathway such as IKK inhibitors;
(y) modulators of cytokine signalling pathyways such as p38 MAP kinase, PI3 kinase, JAK kinase, syk kinase, EGFR or MK-2;
(z) Agents that can be classed as mucolytics or anti-tussive;
(aa) Agents, which enhance responses to inhaled corticosteroids;
(bb) Antibiotics and antiviral agents effective against micro-organisms which can colonise the respiratory tract;
(cc) HDAC inhibitors;
(dd) CXCR2 antagonists;
(ee) Integrin antagonists;
(ff) Chemokines;
(gg) Epithelial sodium channel (ENaC) blockers or Epithelial sodium channel (ENaC) inhibitors;
(hh) P2Y2 Agonists and other Nucleotide receptor agonists;
(ii) Inhibitors of thromboxane;
(jj) Inhibitors of PGD₂ synthesis and PGD₂ receptors (DP1 and DP2/CRTH2);
(kk) Niacin; and
(ll) Adhesion factors including VLAM, ICAM, and ELAM.
